SEQUENCE LISTING (72 sequences)

SEQ ID NO: 1 - DNA, 3069 bp, Homo sapiens
aagcttctagaaggggtatattttgcccccaggccccaaatcctggtctttggactacagtcaagacctagcacagggctcaggcctaaaaaaaagctgttgaggagggtgtgaggctgcaacgttgcggtgagaagggggtccccagggagggcgcagcaggaagccccagggaagtgcctgggagagggaggttgtgcagccagaaggagcagcccgcagcctttggttgttgactcctgctttgtaagtggcagtctggttgggtaggacagggtccgagtcctcactcagggaactgagtccagggtgagctgagcagcccttcctggtggcctcactttcccctgcagagcctgctgcatggtgtccagtggccagcctgggaggagctcaggactggcctcacctcgtgccacgccctcgtaccaaggtggctcagatggcacctgggttcccgaagggcccaggaacacagcgtccatgtccccatccttccccggagggacttggggtggggcctgcaggaaggatcatgtgacttgttcaagggcagcttgtcctgccccggacacagaggtgcccatcgtaagcgagatgcaggcaaggtgaggacagggcatggtgccgagcaggaatgattttcgaaaatgctactctgtggtcgggcacggtggctcactcctgtaatcccagtactttgggaggcccaggcgggcagatcatgaggtcaggagttcaagaccagcctggccaacatggtgaaaccccttctctactgaaaatacaaaaaactacccgggcgtggtggtggatgcctgtaatcccagctactcaggaggctgaggcaggagaatcgcttgaaccctggaggtgaaggttgcaatgagccgagattgcaccactgcactccgacctgggcgacagagaaagactccgtctgaaaaacaaacaaacaaaaccccgagattctcttttcccccgtccggagctctatggccatctggagctcgttctgtccacaaggacacatttcctggcagcagctgtggaccagggctcactcactcacatctgtacccaatctagagcaggacaaacacctatcacctgcactggcaatggacagaggacagtggcagccccatcaccagaggcattcaagccaaggcattttctatcgtctatttatctatctatctatctatctatctatctatctatctatctatctatctagatgagatcttgctatgttgcccaggttgaactcctggcctcaagcgatcctcctgcttcagcctcccaaagtgttgggatttcaggtgtgaaccactgtacctggctggagtgcagtggcactgtcatagctcactgcagcctccacctcctgggctcaaggaatcctctttcctcagcctcctgagtagctgggaccacaggcatgcaccatcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 2 - DNA, 1934 bp, Homo sapiens
gtggcagccccatcaccagaggcattcaagccaaggcattttctatcgtctatttatctatctatctatctatctatctatctatctatctatctatctatctagatgagatcttgctatgttgcccaggttgaactcctggcctcaagcgatcctcctgcttcagcctcccaaagtgttgggatttcaggtgtgaaccactgtacctggctggagtgcagtggcactgtcatagctcactgcagcctccacctcctgggctcaaggaatcctctttcctcagcctcctgagtagctgggaccacaggcatgcaccatcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 3 - DNA, 1619 bp, Homo sapiens
catcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 4 - DNA, 1449 bp, Homo sapiens
gtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 5 - DNA, 1240 bp, Homo sapiens
ggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 6 - DNA, 943 bp, Homo sapiens
gacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 7 - DNA, 660 bp, Homo sapiens
gcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 8 - DNA, 370 bp, Homo sapiens
catccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 9 - DNA, 191 bp, Homo sapiens
gtgagtggggacgctgcccccgcctctcctggtccgtgcacaaagcgcaccccacccgcgcccctccagcctcccgccctttctcgctcgcggcgccgctgcggtccggagcactgcccattgcgcccccgctaggcacgcgggctaccccgcctggagggggcgaggagtggggctttgcccatccgcgg

SEQ ID NO: 10 - DNA, 1896 bp, Homo sapiens
gtgtcagcccttccccccagcccctcctccggcgtctgccccgcgctcgaggccgcggcctttgtgagcaggggggcgggtcgcctcgactgggtcctccctcgccccctttgtcctgctccatctctgcagatgggaaaaccagatgcggcggggcgggggaggggatcggcttttgcggttcacccctgcagaggagccccccgcgccgcccccgggcccagggctcgggtcccgaggttccccaggaggcggtctccctcctcgtcgccgcggcccgggaacggcgtggcgcggatggcggccctccaggcaccccgcccttcgcccgccgcgcgcgtctccaggccggtgcgctgagctccgctccgcgggcgaccgagggcggcttcagcgcgagccgggagaccccaggccagctccctcgggaagcccctaccctctggtgaacccatcccctagggcgctcgccgggaacaattgactgcaactcgatccgtcccctccggggcctcctgcaggcacggctatttgcgaccggcctgtgcgccactctttcccttctttctcagattctccccgaggggctttctcttccttttggcgtcctcttccactcctggcggagaagctgttccttgcttgcagacagaggccccgctggagggaggggcgtggggggatctttttccctggataggaagtcggaccccaggccttggagatggctggacgggagcccagtttgtgtcctcatccctcctcttggaaggagccctcctgacggcccccctcttggctgtgggctctgcaggagggggaaagaccccccatggcagtgggggtgagggtggaggcctgggtggggtaatggccattttgtgcctgaagtttgctaccttaagtcccagagaggtgaagctgcttgtcatgggtcacacagcaagtgaccacaaggaagcagcatgcagaagtgggtgcatttggctccagaaaccccgtttcctgtttcccacagcgcatggcgggcagagctcctcacctgatcagcaggcattgagccctactaaggggacctgctgagccatgagtgaaccagtggggttcacgttctcttggggacattacgaagtgtcaaggaaaaggcagcgggtacaggttattagatggctgagtcctttctgagcaggacatttgagctggcctgaaggatgaatagaaggtggcaggtatggggggggtgctgtgtggaccagcgttgtaggctgtgggtgtctcctgtgcaaaggtcttatggcaggaaggtacagagaaagcccagtgtggctgggaggaagcttgtgaggtctgggccacgggaccttgggtgagagcttagtgcagggctatggtccttgccttggggttgcagccacagcctcccgtggggaagaagtcgcagaagtaggtggccttggccctgggggccaggaagttgcagagagcctgagggtgtttttggcgagcctgggcagataactcccctctcctgagaagcttgctgggggcgtctggtgtgtgattcaggctgatagggtgggaggaaccagaaattgcagccaggagaggtggtggtgatggctcagcttggagttgaaagggatgtgggacggcgcgggggtgggggtacttgggggccgaggttggggctcctctggcctcagatcttggggtccttatctcgtttctatgtcagccaactcccggggggcttttgggaactggagtctgcaggggtgggcgtggggggcgtgtctgcagaggccattttggacgggcctgcgtggcaagggggaggcgtctgcttccgacacacatgctggggaacgccagccaggaaggggaggatccgagctc

SEQ ID NO: 11 - DNA, 356 bp, Homo sapiens
gggcccggtgggggcagggtgctcccctttgaaaaacgctgcgccctgctcttcagcctcagtttccccatctgtaaagtgtgcattccaggcctccttgggctggcccagagctgccctgggcaggggctttcagcccttcacaccagaggctgggtcagctgagtgagtgctcctttgtcctcccgccatgccccaggctcctccctgctccccaggatccacacctaccctggccccgaccctgggccatgcctcaccacacttcctgtcttctctcacatctctcacatctgggagtcctctcccgccagcctgtgcttgcatctggggatccatgccagggataggacctg

SEQ ID NO: 12 - DNA, 550 bp, Homo sapiens
ctgcagtgagccgagatcgtgccactgcactccagcctgggcaacagaacgagaccctgtcttaaaaaaattttttttggcctggtgcggtggctcacgcctgtaatgccagcactttgggagtccaaggtgggtggatcacctggaggtcgggagtttgagaccagtctgaccaacatggagaaaccccatctctactaaaaacacaaaaattagccgggcgtggtggcgcatgcctgtaatcccagctactctggaggctgaggcaggagaatcccttgaacccgggaggcggaagttgcagtgagccgagatcgcaccattgcactccagcctgggcaacaagagtgaaactccatctcaaaaaacaaaacaaaacaaaaaaaacaaccaaaacaaacaaacaaaaaactgtaattaaaataattaaaagaaaaaagttaaaaaaaaaaaaaagagaggatggctcaaaggctggaaacagggaaggccactgtgaggggaggacaggcagggctagacctctctggaaggaggagggagaggtacc

SEQ ID NO: 13 - DNA, 1426 bp, Homo sapiens
gggactgagtcgtggtacagatggcggcagttgtgtggccctggggcccaggaatggctgggaagctccctttcttctctggcttgggggatgaggttagtgtaaatttcatggagttgccaggaggattaaggccgacgtgcattcacacgtggccccgtggctgccgccagttaagccctgcgcagccattggaagtctcattgagaaggagcctcccggcttcctgcatttgttccaggcgggctggtaagcgccctgcttatttgcaaggctgtgtgaggacgagccctgtaaaccctaggacgaggctgtgctaatgtggtttctgttccactgtctccccttctccttttgcccctctttctggggaacctggagcccctgatgccctctgttttttcctgctgctggggtggacctagaacctctccctccatcctttctgctgctggggtgggcctgcagaagactgtcctcttacctgagtccctaacttccctccgatcagcgggggcttcagcggagccccagcgcattggacaggtggtgctctgtggccggcacaggccagggcccagggtgcggcccctgcctgtaggtagacatgcaggcccttctgagcagaatgagggggcggtgagggcagctgtgcgggtgtggctggcctgacggctccctgcagccctgcggcttctgtgcagtgggggtggggcgggccagacctttgcagggcctcttggctggtggagggctgagtgagcagaggccccagccctcgtgttccctggggtgtggccttaggatggtcccggcaccctggggacccagcccctgctcaccttatcctcctcccgtgcttggctggggtgtggtggctgagccagcccacccagtgcggtggatgctcgtcggagggcagagggtgggctaccggcttaaggggccccagggaacctggggggtggagcccaaggggttccaagaaggggcggggccaggaacccatagacaagagggtggggcagggaaagggcgtggcaagagggccacccacctccggtccagagagccagccagacccttactttctcttgctgtgtgaccttaggcaaggacatgccaccaccggccttagtctccctctttgtgaagtaggatgaagatccccacctgtggagcagatgtggggctgcatggttgggtcaggatggaacaggatggggaggccgggcgtggtggcttacccctgtaatcccagcactttgggaggcaaaggtgggaggactgcttgagtcctggagtttgggcaacatagcgagaccacccccatctctacaaaataatgttaaagttagccaggtgtagtggtgagtgcctgtggtcccacctactggggagactgaggcaagaggatcctttgagc

SEQ ID NO: 14 - DNA, 477 bp, Homo sapiens
ctgcagcctccgcctcctgggttcaagcaattctcctgcgtcagcctcccgagcagctgggattacaggcgcccggcaccacccccagctaatttttttttttttgagacagagtctcgcattgtcgcccaggctggagtgcagtggcgcgatcttggctcactgcaagctccacctcccgggttcacgccattctcctgcctcagcctcccgagtagcagggactacaggcgcccgccaccgtgcccggctaattttttttttttgtattattaatagagacgggtttcaccgtgttaggatgatctcgatctcctgacctcgtgatccgcctgcctcggcctcccaaagtgctgggattacaggcatgagccaccttgcctggtccttttttttgtatttttagtagagatgaggtttcaccatgttggccaggctggtctcaaactcctgacctcatgatccgcccgcctcggc

SEQ ID NO: 15 - DNA, 473 bp, Homo sapiens
ctgcagaacacccctgtgttggcaaatacatctctttctttttgattcagggtcttgctctgttgcccaggttggagtgcagtggcacaatcatagctcactgtagcctcgacttcctgggctcaagtgatcctcccacctcagcctcccaaatagctgagactacaggcacacaccaccacatcaagttaatttttttttctatttggtacagacagggtctggctatgttgcctaggatggtcttgaactcccggcctcacatgatcctcctaactcagcctcccaaagtgctgaggttacagacacaagccaccatgtgcccagccagtgaacccacaactttctagctgtttttttcttatacataagaagcatgcgggttgccacggcggctcacgcctgtaatcccagcactttgggaagctgaggcgggtggatcacctgaggtcatgagttcaagaccagccttg

SEQ ID NO: 16 - DNA, 311 bp, Homo sapiens
caatcctcctgccttgggcctcccaaagtgttgcgatcacaggcatgagcccctgtccctggcccctattatttctctaactggggaaagtcatgtgggaacagatgtagcttgccttggcctctgaccggccctgcctgcgttcctgggtgctctctgctgcttctcatgtgtgccactgtggggactcggccgcccaccccaccccgtggtgttaccttgcgtgtgtagttctgtgagctcagggctatggtctgccagaactagggggcgtggggccccagtaccagcccaaggcctcctctctgcag

SEQ ID NO: 17 - DNA, 82 bp, Homo sapiens
gtgagtgcctcgctcccacctgtcaccgcccccaccaccttgcctgggctaccccgcctgaccctgtcccgccatcccccag

SEQ ID NO: 18 - DNA, 245 bp, Homo sapiens
gtaacactaaagccccagttccctggagccgtcctggagtcctggagggtctggccatgccgtggtcacttggtagccccagccaaggcctgctctgtgctgggcatccccccggactggccccgcactgtcctaccctggggactgctgtgtgaccccagctcctggccctccctctctggtcaccccagcctccaccccactccctgccaggaggctcactgactcccctctttctgggacag

SEQ ID NO: 19 - DNA, 1242 bp, Homo sapiens
gcaggttctcctgtgggcagctgctgggcagggaacccctcggtcggggctggggtcagtgctgcggggagcgccctctgcatccacactggaccctggcttggctcagggccattccaggccctaaagggacaggtgtctgatggccaccagggggctctgggatgcaagcagcccctttccctcttgtctgtgtggttggggggacttaccttgcccacctgacagagaggtgtgtggaggggagagcagggagggaaggagaccaggaaagggagggaggagagcaggggaggggagagccgggaagagaggagagcaggggtggggaggtttctggaaagggtgtgcaaggggaggacgcgcctcggttatgggactggagccccttcccaggaggacccccaacaatccagaggtgcctgttaggattcagaacatggtttttttgtttgtttttttgagactcactccctcaccctggctggacttgcagtggcgttatctcggctcactgcaacctctgcctcctgggttcaagtgattctcctgcctcagcctcccaagtagctgggattacaggtgtgcaccaccacgcctggctaatttttatatttttaaaatttattatttatttattttgagacccagtctggagtgcagtggcgttatctcggctctctgcaacctctgcctcctgggttcaagcgattctcctgcctcagcctcccgagtagctgggactatgtgtgggagccaccatgcctggctaatttttttgtatttttcatagagacgggtttcaccatgttgtccaggctggtcttgaattcgtggcctcaagtgatccgcccacctcagcctcccacagtgctgggtttataggtgtgagccaccacacccggctaattgttttgtatttttagtagagacggagcttcactatgttggcaaggctggctcgaactcctgacctcaagtgatccgcccacctcagcctcccaaagtgctgggattacaggcgtgagccactgcggccgagcagaacacgttctaggacccttgttcatgtgtccatcatggacaggaggacgtgcgggccatagggaccctggctcattccggagccgggactggagggtggggcgtcacccttgggaacacccgtgcccaccctccgctgcccagggtaggggtggggagccaggctttgggccccacttgataaagtcccctccccag

SEQ ID NO: 20 - DNA, 161 bp, Homo sapiens
agccccaggccggcctgtgtgtgtcttggggctgaggtgggtgggggggctgaggtgggtgggagggctggcgggacaggtaggcgccctggctccccagctcagtgctgggagtgtgcagtgggagggaggccgtggctccagtgggtgctccggagctc

SEQ ID NO: 21 - DNA, 211 bp, Homo sapiens
cctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 22 - DNA, 155 bp, Homo sapiens
cacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtct

SEQ ID NO: 23 - DNA, 20 bp, Artificial Sequence
  Description of Artificial Sequence Synthetic primer
gctgacctgctggattacat

SEQ ID NO: 24 - DNA, 22 bp, Artificial Sequence
  Description of Artificial Sequence Synthetic primer
cattatagtcaagggcatatcc

SEQ ID NO: 25 - DNA, 20 bp, Artificial Sequence
  Description of Artificial Sequence Synthetic primer
ggcaagcctggctgtagtag

SEQ ID NO: 26 - DNA, 21 bp, Artificial Sequence
  Description of Artificial Sequence Synthetic primer
ccagagtgagatgcatgttgg

SEQ ID NO: 27 - DNA, 2772 bp, Homo sapiens
  CDS (112)..(1590)
gcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacggtgtcccccgccgagaagtggagcgtgcacatcgccatgcaccctcaggtcaacatctacagtgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagaatgccagctgctactttgacatcgagtggcgtgaccggcgcatcacactgagggcgtccaatggcaagtttgtgacctccaagaagaatgggcagctggccgcctcggtggagacagcaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaagactccacaggcaaatactggacggtgggcagtgactccgcggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctgagccttatttctctggaagcggctaagggacggttgggggctgggagccctgggcgtgtagtgtaactggaatcttttgcctctcccagccacctcctcccagccccccaggagagctgggcacatgtcccaagcctgtcagtggccctccctggtgcactgtccccgaaacccctgcttgggaagggaagctgtcgggagggctaggactgacccttgtggtgtttttttgggtggtggctggaaacagcccctctcccacgtgggagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggcccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcgcgtg

SEQ ID NO: 28 - protein, 493 aa, Homo sapiens
MTANGTAEAVQIQFGLINCGNKYLTAEAFGFKVNASASSLKKKQIWTLEQPPDEAGSAAVCLRSHLGRYLAADKDGNVTCEREVPGPDCRFLIVAHDDGRWSLQSEAHRRYFGGTEDRLSCFAQTVSPAEKWSVHIAMHPQVNIYSVTRKRYAHLSARPADEIAVDRDVPWGVDSLITLAFQDQRYSVQTADHRFLRHDGRLVARPEPATGYTLEFRSGKVAFRDCEGRYLAPSGPSGTLKAGKATKVGKDELFALEQSCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 29 - DNA, 2634 bp, Mus musculus
  CDS (79)..(1557)
gagctaagccgtgttgaacaaaggaggtcgggcacagctatccaagctcccggggccaccgggccgccctccgccaccatgaccgccaacggcacggcagaggctgtgcagattcagttcgggctcatcagctgcggcaacaagtacctgacagccgaggcgttcgggttcaaggtgaacgcatccgctagtagcttgaaaaagaagcagatctggacgctggagcaacctcccgatgaggcgggcagcgcggccgtgtgtctgcgcacgcacctgggtcgctacctggccgccgacaaggacggcaacgtgacctgcgagcgcgaggtgcccgacggcgactgccgctttctcgtcgtggcgcacgacgacggccgctggtcgctgcagtccgaggctcaccggcgctactttggcggcaccgaggaccgcctgtcctgcttcgcgcagagcgtgtcgccggccgagaagtggagcgtgcacatcgccatgcacccgcaggttaacatctacagcgttacccgcaagcgctacgcgcatctgagcgcgcggccggccgacgagatcgcggtagaccgcgacgtgccttggggcgtcgactcgctcatcaccttggccttccaggaccaacgctacagtgtgcagacgtccgaccaccgcttcctgcgccacgacgggcgccttgtggcacggccggagcccgccacgggcttcacgctggagttccgctccggcaaggtggcctttcgcgactgcgaaggtcgctacctggctccgtccgggcccagcggcaccctcaaggctggcaaggccaccaaggtgggcaaagatgagctcttcgccctggaacagagctgcgctgaggtggtgctgcaggcggccaacgaggggaacgtgtccacgcgccagggaatggacctgtcagccaatcaggatgaagagaccgatcaggagaccttccagctggagatcgaccgcgacacaagaaagtgtgcctttcgcacccacacgggcaagtactggacactgacggcgaccggaggtgtgcaatccactgcgtccaccaagaacgccagctgctactttgacatcgagtggtgtgaccgccggatcactctgagagcctccaacggcaagtttgtgaccgccaagaaaaatggccacgtggccgcctcggtggagacagcaggggactcggaactcttcctcatgaagctgattaaccgccccatcattgcgttccggggggaacacgggttcattgcgtgccgcaaggtcacgggcactctggatgccaaccgttccagttacgatgtcttccagttggaattcaatgacggcgcctacaacatcaaagactccacgggcaagtactggacggtgggtagtgattcctcggtcaccagcagcagcgacacccctgtggatttcttccttgagttctgtgactacaataaggtggctctcaaggtgggcggccgctacctgaagggggaccacgctggggtcctgaaggcctgcgcggagactatcgaccccgcctcactctgggagtactagggccacctgcctctgcagccgctctcgtcagtcctcctgttatccttactcatcgggtggcctgcagcaggtggcaaaccccttgcctttcaaactggaaacccaagagaaaacggtgcccttgctgtcaccctctgtggaccccttttccctaactcactgctccccatgggtcggtggctgcagactgtccccaggaggactctggttccctctgtccccttctttccatggggaactctggcacctttcttctgacctcagtcaactctgagccttatttccccccaggaagtggcctaggagaagctacagggcctagggacttaccctgagcttgtaactggaagaccccgtccctatccccgctcccgcccccaccccaccccacccctgctctggccccagcctctggaggccagccttttggcgggactgaagccgggcatggccaaccttgcccacaagtgtttttctggatcttggctggaaggcagtctgtcccatcctgcagtgtttgggcctggctctttgactcaaagctagctaggtggcactccgtgtcgctcctgcacattctggaaggggcgggcctctcacccacctcattccttttccccctggcctgactggaagcagaaaaatgaccaaatcagtattttttttttttctttaaggaaatgttactgttgaaaggccctaggcaagcctgccctgttggttgtagtcgtgagtggtcttggggggagatgcttggctcctgtccctgcctccccagcggttccctccctccctcctgcctgaccaccccagctctggctctgtgattggtgctccacgtctccagacacctcggggctcctgggcggagaaagccgatgtgcccctccctgggagccctgagtaaacctcagggggccctttcccaatcacccctccaccgacccctcaacaccatgcatctcactctgggtgtactcgctcacatttatttttttgtaactgtcatttctataactctgaagacccatgatagtaagctttgaactggaaaataaagtaaaatcaagtctg

SEQ ID NO: 30 - protein, 493 aa, Mus musculus
MTANGTAEAVQIQFGLISCGNKYLTAEAFGFKVNASASSLKKKQIWTLEQPPDEAGSAAVCLRTHLGRYLAADKDGNVTCEREVPDGDCRFLVVAHDDGRWSLQSEAHRRYFGGTEDRLSCFAQSVSPAEKWSVHIAMHPQVNIYSVTRKRYAHLSARPADEIAVDRDVPWGVDSLITLAFQDQRYSVQTSDHRFLRHDGRLVARPEPATGFTLEFRSGKVAFRDCEGRYLAPSGPSGTLKAGKATKVGKDELFALEQSCAEVVLQAANEGNVSTRQGMDLSANQDEETDQETFQLEIDRDTRKCAFRTHTGKYWTLTATGGVQSTASTKNASCYFDIEWCDRRITLRASNGKFVTAKKNGHVAASVETAGDSELFLMKLINRPIIAFRGEHGFIACRKVTGTLDANRSSYDVFQLEFNDGAYNIKDSTGKYWTVGSDSSVTSSSDTPVDFFLEFCDYNKVALKVGGRYLKGDHAGVLKACAETIDPASLWEY

SEQ ID NO: 31 - DNA, 20 bp, Artificial Sequence
  Description of Artificial Sequence Synthetic primer
gccaccatgaccgccaacgg

SEQ ID NO: 32 - DNA, 20 bp, Artificial Sequence
  Description of Artificial Sequence Synthetic primer
tgtgtcgcggtcgatctcca

SEQ ID NO: 33 - DNA, 4100 bp, Homo sapiens
aagcttctagaaggggtatattttgcccccaggccccaaatcctggtctttggactacagtcaagacctagcacagggctcaggcctaaaaaaaagctgttgaggagggtgtgaggctgcaacgttgcggtgagaagggggtccccagggagggcgcagcaggaagccccagggaagtgcctgggagagggaggttgtgcagccagaaggagcagcccgcagcctttggttgttgactcctgctttgtaagtggcagtctggttgggtaggacagggtccgagtcctcactcagggaactgagtccagggtgagctgagcagcccttcctggtggcctcactttcccctgcagagcctgctgcatggtgtccagtggccagcctgggaggagctcaggactggcctcacctcgtgccacgccctcgtaccaaggtggctcagatggcacctgggttcccgaagggcccaggaacacagcgtccatgtccccatccttccccggagggacttggggtggggcctgcaggaaggatcatgtgacttgttcaagggcagcttgtcctgccccggacacagaggtgcccatcgtaagcgagatgcaggcaaggtgaggacagggcatggtgccgagcaggaatgattttcgaaaatgctactctgtggtcgggcacggtggctcactcctgtaatcccagtactttgggaggcccaggcgggcagatcatgaggtcaggagttcaagaccagcctggccaacatggtgaaaccccttctctactgaaaatacaaaaaactacccgggcgtggtggtggatgcctgtaatcccagctactcaggaggctgaggcaggagaatcgcttgaaccctggaggtgaaggttgcaatgagccgagattgcaccactgcactccgacctgggcgacagagaaagactccgtctgaaaaacaaacaaacaaaaccccgagattctcttttcccccgtccggagctctatggccatctggagctcgttctgtccacaaggacacatttcctggcagcagctgtggaccagggctcactcactcacatctgtacccaatctagagcaggacaaacacctatcacctgcactggcaatggacagaggacagtggcagccccatcaccagaggcattcaagccaaggcattttctatcgtctatttatctatctatctatctatctatctatctatctatctatctatctatctagatgagatcttgctatgttgcccaggttgaactcctggcctcaagcgatcctcctgcttcagcctcccaaagtgttgggatttcaggtgtgaaccactgtacctggctggagtgcagtggcactgtcatagctcactgcagcctccacctcctgggctcaaggaatcctctttcctcagcctcctgagtagctgggaccacaggcatgcaccatcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacgggtcccccgccgagaagtggagcgtgcacatcgccatgcaccctcaggtcaacatctacagcgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtgagtggggacgctgcccccgcctctcctggtccgtgcacaaagcgcaccccacccgcgcccctccagcctcccgccctttctcgctcgcggcgccgctgcggtccggagcactgcccattgcgcccccgctaggcacgcgggctaccccgcctggagggggcgaggagtggggctttgcccatccgcgg

SEQ ID NO: 34 - DNA, 2843 bp, Homo sapiens
caatcctcctgccttgggcctcccaaagtgttgcgatcacaggcatgagcccctgtccctggcccctattatttctctaactggggaaagtcatgtgggaacagatgtagcttgccttggcctctgaccggccctgcctgcgttcctgggtgctctctgctgcttctcatgtgtgccactgtggggactcggccgcccaccccaccccgtggtgttaccttgcgtgtgtagttctgtgagctcagggctatggtctgccagaactagggggcgtggggccccagtaccagcccaaggcctcctctctgcaggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagtgagtgcctcgctcccacctgtcaccgcccccaccaccttgcctgggctaccccgcctgaccctgtcccgccatcccccaggaatgccagctgctactttgacatcgagtggcgtgaccggcgcatcacactgagggcgtccaatggcaagtttgtgacctccaagaagaatgggcagctggccgcctcggtggagacagcaggtaacactaaagccccagttccctggagccgtcctggagtcctggagggtctggccatgccgtggtcacttggtagccccagccaaggcctgctctgtgctgggcatccccccggactggccccgcactgtcctaccctggggactgctgtgtgaccccagctcctggccctccctctctggtcaccccagcctccaccccactccctgccaggaggctcactgactcccctctttctgggacaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaaggcaggttctcctgtgggcagctgctgggcagggaacccctcggtcggggctggggtcagtgctgcggggagcgccctctgcatccacactggaccctggcttggctcagggccattccaggccctaaagggacaggtgtctgatggccaccagggggctctgggatgcaagcagcccctttccctcttgtctgtgtggttggggggacttaccttgcccacctgacagagaggtgtgtggaggggagagcagggagggaaggagaccaggaaagggagggaggagagcaggggaggggagagccgggaagagaggagagcaggggtggggaggtttctggaaagggtgtgcaaggggaggacgcgcctcggttatgggactggagccccttcccaggaggacccccaacaatccagaggtgcctgttaggattcagaacatggtttttttgtttgtttttttgagactcactccctcaccctggctggacttgcagtggcgttatctcggctcactgcaacctctgcctcctgggttcaagtgattctcctgcctcagcctcccaagtagctgggattacaggtgtgcaccaccacgcctggctaatttttatatttttaaaatttattatttatttattttgagacccagtctggagtgcagtggcgttatctcggctctctgcaacctctgcctcctgggttcaagcgattctcctgcctcagcctcccgagtagctgggactatgtgtgggagccaccatgcctggctaatttttttgtatttttcatagagacgggtttcaccatgttgtccaggctggtcttgaattcgtggcctcaagtgatccgcccacctcagcctcccacagtgctgggtttataggtgtgagccaccacacccggctaattgttttgtatttttagtagagacggagcttcactatgttggcaaggctggctcgaactcctgacctcaagtgatccgcccacctcagcctcccaaagtgctgggattacaggcgtgagccactgcggccgagcagaacacgttctaggacccttgttcatgtgtccatcatggacaggaggacgtgcgggccatagggaccctggctcattccggagccgggactggagggtggggcgtcacccttgggaacacccgtgcccaccctccgctgcccagggtaggggtggggagccaggctttgggccccacttgataaagtcccctccccagactccacaggcaaatactggacggtgggcagtgactccgcggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctga

SEQ ID NO: 35 - DNA, 934 bp, Homo sapiens
ccacctcctcccagccccccaggagagctgggcacatgtcccaagcctgtcagtggccctccctggtgcactgtccccgaaacccctgcttgggaagggaagctgtcgggtgggctaggactgacccttgtggtgtttttttgggtggtggctggaaacagcccctctcccacgtggcagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggcccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcagccccaggccggcctgtgtgtgtcttggggctgaggtgggtgggggggctgaggtgggtgggagggctggcgggacaggtaggcgccctggctccccagctcagtgctgggagtgtgcagtgggagggaggccgtggctccagtgggtgctccggagctc

SEQ ID NO: 36 - DNA, 20 bp, Artificial Sequence
  Description of Artificial Sequence Synthetic primer
aagcttctagaaggggtata

SEQ ID NO: 37 - DNA, 20 bp, Artificial Sequence
  Description of Artificial Sequence Synthetic primer
agacgagaggccgctgcgcg

SEQ ID NO: 38 - DNA, 20 bp, Artificial Sequence
  Description of Artificial Sequence Synthetic primer
gtggcagccccatcaccaga

SEQ ID NO: 39 - DNA, 20 bp, Artificial Sequence
  Description of Artificial Sequence Synthetic primer
catcacacccagctaaacct

SEQ ID NO: 40 - DNA, 20 bp, Artificial Sequence
  Description of Artificial Sequence Synthetic primer
gtgcgggttctggggctcaa

SEQ ID NO: 41 - DNA, 20 bp, Artificial Sequence
  Description of Artificial Sequence Synthetic primer
ggcagcctgtggtcatgctg

SEQ ID NO: 42 - DNA, 20 bp, Artificial Sequence
  Description of Artificial Sequence Synthetic primer
gacctgggctggtctggctc

SEQ ID NO: 43 - DNA, 20 bp, Artificial Sequence
  Description of Artificial Sequence Synthetic primer
gcagggcctcgggagacaac

SEQ ID NO: 44 - DNA, 20 bp, Artificial Sequence
  Description of Artificial Sequence Synthetic primer
catccgggggggttaggtgg

SEQ ID NO: 45 - DNA, 18 bp, Artificial Sequence
  Description of Artificial Sequence Synthetic primer
gtgagtggggacgctgcc

SEQ ID NO: 46 - DNA, 18 bp, Artificial Sequence
  Description of Artificial Sequence Synthetic primer
ccgcggatgggcaaagcc

SEQ ID NO: 47 - DNA, 20 bp, Artificial Sequence
  Description of Artificial Sequence Synthetic primer
gtgtcagcccttccccccag

SEQ ID NO: 48 - DNA, 20 bp, Artificial Sequence
  Description of Artificial Sequence Synthetic primer
gagctcggatcctccccttc

SEQ ID NO: 49 - DNA, 20 bp, Artificial Sequence
  Description of Artificial Sequence Synthetic primer
gggcccggtgggggcagggt

SEQ ID NO: 50 - DNA, 20 bp, Artificial Sequence
  Description of Artificial Sequence Synthetic primer
caggtcctatccctggcatg

SEQ ID NO: 51 - DNA, 20 bp, Artificial Sequence
  Description of Artificial Sequence Synthetic primer
ctgcagtgagccgagatcgt

SEQ ID NO: 52 - DNA, 20 bp, Artificial Sequence
  Description of Artificial Sequence Synthetic primer
ggtacctctccctcctcctt

SEQ ID NO: 53 - DNA, 20 bp, Artificial Sequence
  Description of Artificial Sequence Synthetic primer
gggactgagtcgtggtacag

SEQ ID NO: 54 - DNA, 20 bp, Artificial Sequence
  Description of Artificial Sequence Synthetic primer
gctcaaaggatcctcttgcc

SEQ ID NO: 55 - DNA, 20 bp, Artificial Sequence
  Description of Artificial Sequence Synthetic primer
ctgcagcctccgcctcctgg

SEQ ID NO: 56 - DNA, 20 bp, Artificial Sequence
  Description of Artificial Sequence Synthetic primer
gccgaggcgggcggatcatg

SEQ ID NO: 57 - DNA, 20 bp, Artificial Sequence
  Description of Artificial Sequence Synthetic primer
ctgcagaacacccctgtgtt

SEQ ID NO: 58 - DNA, 20 bp, Artificial Sequence
  Description of Artificial Sequence Synthetic primer
caaggctggtcttgaactca

SEQ ID NO: 59 - DNA, 20 bp, Artificial Sequence
  Description of Artificial Sequence Synthetic primer
caatcctcctgccttgggcc

SEQ ID NO: 60 - DNA, 20 bp, Artificial Sequence
  Description of Artificial Sequence Synthetic primer
ctgcagagaggaggccttgg

SEQ ID NO: 61 - DNA, 20 bp, Artificial Sequence
  Description of Artificial Sequence Synthetic primer
gtgagtgcctcgctcccacc

SEQ ID NO: 62 - DNA, 20 bp, Artificial Sequence
  Description of Artificial Sequence Synthetic primer
ctgggggatggcgggacagg

SEQ ID NO: 63 - DNA, 20 bp, Artificial Sequence
  Description of Artificial Sequence Synthetic primer
gtaacactaaagccccagtt

SEQ ID NO: 64 - DNA, 20 bp, Artificial Sequence
  Description of Artificial Sequence Synthetic primer
ctgtcccagaaagaggggag

SEQ ID NO: 65 - DNA, 20 bp, Artificial Sequence
  Description of Artificial Sequence Synthetic primer
gcaggttctcctgtgggcag

SEQ ID NO: 66 - DNA, 20 bp, Artificial Sequence
  Description of Artificial Sequence Synthetic primer
ctggggaggggactttatca

SEQ ID NO: 67 - DNA, 20 bp, Artificial Sequence
  Description of Artificial Sequence Synthetic primer
agccccaggccggcctgtgt

SEQ ID NO: 68 - DNA, 20 bp, Artificial Sequence
  Description of Artificial Sequence Synthetic primer
gagctccggagcacccactg

SEQ ID NO: 69 - DNA, 25 bp, Artificial Sequence
  Description of Artificial Sequence Synthetic primer
aagcttctagaaggggtatattttg

SEQ ID NO: 70 - DNA, 19 bp, Artificial Sequence
  Description of Artificial Sequence Synthetic primer
cgttcaccttgaacccgaa

SEQ ID NO: 71 - DNA, 21 bp, Artificial Sequence
  Description of Artificial Sequence Synthetic primer
gcagccgaacaaaggagcagg

SEQ ID NO: 72 - DNA, 16951 bp, Homo sapiens
aagcttctagaaggggtatattttgcccccaggccccaaatcctggtctttggactacagtcaagacctagcacagggctcaggcctaaaaaaaagctgttgaggagggtgtgaggctgcaacgttgcggtgagaagggggtccccagggagggcgcagcaggaagccccagggaagtgcctgggagagggaggttgtgcagccagaaggagcagcccgcagcctttggttgttgactcctgctttgtaagtggcagtctggttgggtaggacagggtccgagtcctcactcagggaactgagtccagggtgagctgagcagcccttcctggtggcctcactttcccctgcagagcctgctgcatggtgtccagtggccagcctgggaggagctcaggactggcctcacctcgtgccacgccctcgtaccaaggtggctcagatggcacctgggttcccgaagggcccaggaacacagcgtccatgtccccatccttccccggagggacttggggtggggcctgcaggaaggatcatgtgacttgttcaagggcagcttgtcctgccccggacacagaggtgcccatcgtaagcgagatgcaggcaaggtgaggacagggcatggtgccgagcaggaatgattttcgaaaatgctactctgtggtcgggcacggtggctcactcctgtaatcccagtactttgggaggcccaggcgggcagatcatgaggtcaggagttcaagaccagcctggccaacatggtgaaaccccttctctactgaaaatacaaaaaactacccgggcgtggtggtggatgcctgtaatcccagctactcaggaggctgaggcaggagaatcgcttgaaccctggaggtgaaggttgcaatgagccgagattgcaccactgcactccgacctgggcgacagagaaagactccgtctgaaaaacaaacaaacaaaaccccgagattctcttttcccccgtccggagctctatggccatctggagctcgttctgtccacaaggacacatttcctggcagcagctgtggaccagggctcactcactcacatctgtacccaatctagagcaggacaaacacctatcacctgcactggcaatggacagaggacagtggcagccccatcaccagaggcattcaagccaaggcattttctatcgtctatttatctatctatctatctatctatctatctatctatctatctatctatctagatgagatcttgctatgttgcccaggttgaactcctggcctcaagcgatcctcctgcttcagcctcccaaagtgttgggatttcaggtgtgaaccactgtacctggctggagtgcagtggcactgtcatagctcactgcagcctccacctcctgggctcaaggaatcctctttcctcagcctcctgagtagctgggaccacaggcatgcaccatcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcgggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggctttgtggagcgctgcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacggtgtcccccgccgagaagtggagcgtgcacatcgccatgcaccctcaggtcaacatctacagcgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtgagtggggacgctgcccccgcctctcctggtccgtgcacaaagcgcaccccacccgcgcccctccagcctcccgccctttctcgctcgcggcgccgctgcggtccggagcactgcccattgcgcccccgctaggcacgcgggctaccccgcctggagggggcgaggagtggggctttgcccatcctcgggtgccgctgcccacctcccaccccgggctgggatcatgggctcccctaggccccgcggagtcgcaaccgtacgtgcaccctcctaacccccccccccgcccaatcttggctctccccacgcgcgctgatccctcggatcagctgtcccagctcttgcggagtgggagcccctcacccatttcctcgtgcccctccccccgccggctgtcctggagctcgggggtccgaggcaagggtcgccacccgcaagggcgcgcctccacccccaccggcagcctttcgcgggcgagatgggggaggttagccaggcctttgatcccggcggggcgcgcctccacctccccgtctgcccggccttcctccacctcctcccgctgccgggcggggtcggcctccgctggtgggggggggcgcggggtgtcagcccttccccccagcccctcctcccgcgtctgccccgcgctcgaggccgcggcctttgtgagcaggggggcgggtcgcctcgactgggtcctccctcgccccctttgtcctgctccatctctgcagatgggaaaaccagatgcggcggggcgggggaggggatcggcttttgcggttcacccctgcagaggagccccccgcgccgcccccgggcccagggctcgggtcccgaggttccccaggaggcggtctccctcctcgtcgccgcggcccgggaacggcgtggcgcggatggcggccctccaggcaccccgcccttcgcccgccgcgcgcgtctccaggccggtgcgctgagctccgctccgcgggcgaccgagggcggcttcagcgcgagccgggagaccccaggccagctccctcgggaagcccctaccctctggtgaacccatcccctagggcgctcgccgggaacaattgactgcaactcgatccgtcccctccggggcctcctgcaggcacggctatttgcgaccggcctgtgcgccactctttcccttctttctcagattctccccgaggggctttctcttccttttggcgtcctcttccactcctggcggagaagctgttccttgcttgcagacagaggccccgctggagggaggggcgtggggggatctttttccctggataggaagtcggaccccaggccttggagatggctggacgggagcccagtttgtgtcctcatccctcctcttggaaggagccctcctgacggcccccctcttggctgtgggctctgcaggagggggaaagaccccccatggcagtgggggtgagggtggaggcctgggtggggtaatggccattttgtgcctgaagtttgctaccttaagtcccagagaggtgaagctgcttgtcatgggtcacacagcaagtgaccacaaggaagcagcatgcagaagtgggtgcatttggctccagaaaccccgtttcctgtttcccacagcgcatggcgggcagagctcctcacctgatcagcaggcattgagccctactaaggggacctgctgagccatgagtgaaccagtggggttcacgttctcttggggacattacgaagtgtcaaggaaaaggcagcgggtacaggttattagatggctgagtcctttctgagcaggacatttgagctggcctgaaggatgaatagaaggtggcaggtatggggggggtgctgtgtggaccagcgttgtaggctgtgggtgtctcctgtgcaaaggtcttatggcaggaaggtacagagaaagcccagtgtggctgggaggaagcttgtgaggtctgggccacgggaccttgggtgagagcttagtgcagggctatggtccttgccttggggttgcagccacagcctcccgtggggaagaagtcgcagaagtaggtggccttggccctgggggccaggaagttgcagagagcctgagggtgtttttggcgagcctgggcagataactcccctctcctgagaagcttgctgggggcgtctggtgtgtgattcaggctgatagggtgggaggaaccagaaattgcagccaggagaggtggtggtgatggctcagcttggagttgaaagggatgtgggacggcgcgggggtgggggtacttgggggccgaggttggggctcctctggcctcagatcttggggtccttatctcgtttctatgtcagccaactcccggggggcttttgggaactggagtctgcaggggtgggcgtggggggcgtgtctgcagaggccattttggacgggcctgcgtggcaagggggaggcgtctgcttccgacacacatgctggggaacgccagccaggaaggggaggatccggacttagctggcaggggggatgtgaatcatctctgcaggcctgcacgggcgggcccggtgggggcagggtgctcccctttgaaaaacgctgcgccctgctcttcagcctcagtttccccatctgtaaagtgtgcattccaggcctccttgggctggcccagagctgccctgggcaggggctttcagcccttcacaccagaggctgggtcagctgagtgagtgctcctttgtcctcccgccatgccccaggctcctccctgctccccaggatccacacctaccctggccccgaccctgggccatgcctcaccacacttcctgtcttctctcacatctctcacatctgggagtcctctcccgccagcctgtgcttgcatctggggatccatgccagggataggacctgtcctccctgctggcttgggacatgccctctcccagccactctgaggagggctgactgaggaagggctcgtcaagctgggctttgcaggatgtgtaagagttctccccacgaggcgcagggcattctgagcccagggaatggcttgtataaggatgcagaggcatttgaaatggccaagtagttgcaggaatcgactggaaaccggggtggtaaggtgaagccatagaacattccaggccccctcccctaaatgagatggaaggagtgcctgttttgaacaagccagggcatctggggaccgttaaggcctgggggtggtgatggggactggagggtgtgaggcaaccagggggtgcccctctgagccaccacagacagaatggttcagaaggccaggcacactggctctcgcctgtaatccaagcactttgggaggccaaggagggaagatggcttcggccaaggagggaagatggcttcagcccaggagttcaaggccagcctggaaaacatggcaaaacctctgtctacaaaaaatacaaaaaattagccaggcatggtgatgcacgcctgtcgtcccagctactcaggaggcttagatgggaggatcacttgagccggggagtttgaggctgcagtgagccgagatcgtgccactgcactccagcctgggcaacagaacgagaccctgtcttaaaaaaattttttttggcctggtgcggtggctcacgcctgtaatgccagcactttgggagtccaaggtgggtggatcacctggaggtcgggagtttgagaccagtctgaccaacatggagaaaccccatctctactaaaaacacaaaaattagccgggcgtggtggcgcatgcctgtaatcccagctactctggaggctgaggcaggagaatcccttgaacccgggaggcggaagttgcagtgagccgagatcgcaccattgcactccagcctgggcaacaagagtgaaactccatctcaaaaaacaaaacaaaacaaaaaaaacaaccaaaacaaacaaacaaaaaactgtaattaaaataattaaaagaaaaaagttaaaaaaaaaaaaaagagaggatggctcaaaggctggaaacagggaaggccactgtgaggggaggacaggcagggctagacctctctggaaggaggagggagaggtacccgtgggcccggcacaggagactcttaatctcctggctcccgggggcttctgtgggcctgtgactcaggatttctgtgcctctgttgatgaaaagaaaaatcctgaagaaaacactgttcccatagtaacacagctctaattagagactccaaggccaagcgagggccttggagccaggaggggccctgctctactcgggggacgacacccctttcccctatccctccctgctggggtgtggcctgatgtctacgtggcaccaggcccctaaatcctttcaacatcctggcggagcggagaggctgggcccttttgacaggtggggaaactgaggaaggttgcagggaaggcactcatccaagggtgcctggccccccgcggtgggtgcctcactggctggcctaaacctttgcatcaaaccagttccaggattgaacgcaacaggctgatggggactgagtcgtggtacagatggcggcagttgtgtggccctgggcccaggaatggctgggaagctccctttcttctctggcttgggggatgaggttagtgtaaatttcatggagttgccaggaggattaaggccgacgtgcattcacacgtggccccgtggctgccgccagttaagccctgcgcagccattggaagtctcattgagaaggagcctcccggcttcctgcatttgttccaggcgggctggtaagcgccctgcttatttgcaaggctgtgtgaggacgagccctgtaaaccctaggacgaggctgtgctaatgtggtttctgttccactgtctccccttctccttttgcccctctttctggggaacctggagcccctgatgccctctgttttttcctgctgctggggtggacctagaacctctccctccatcctttctgctgctggggtgggcctgcagaagactgtcctcttacctgagtccctaacttccctccgatcagcgggggcttcagcggagccccagcgcattggacaggtggtgctctgtggccggcacaggccagggcccagggtgcggcccctgcctgtaggtagacatgcaggcccttctgagcagaatgagggggcggtgagggcagctgtgcgggtgtggctggcctgacggctccctgcagccctgcggcttctgtgcagtgggggtggggcgggccagacctttgcagggcctcttggctggtggagggctgagtgagcagaggccccagccctcgtgttccctggggtgtggccttaggatggtcccggcaccctggggacccagcccctgctcaccttatcctcctcccgtgcttggctggggtgtggtggctgagccagcccacccagtgcggtggatgctcgtcggagggcagagggtgggctaccggcttaaggggccccagggaacctggggggtggagcccaaggggttccaagaaggggcggggccaggaacccatagacaagagggtggggcagggaaagggcgtggcaagagggccacccacctccggtccagagagccagccagacccttactttctcttgctgtgtgaccttaggcaaggacatgccaccaccggccttagtctccctctttgtgaagtaggatgaagatccccacctgtggagcagatgtggggctgcatggttgggtcaggatggaacaggatggggaggccgggcgtggtggcttacccctgtaatcccagcactttgggaggcaaaggtgggaggactgcttgagtcctggagtttgggcaacatagcgagaccacccccatctctacaaaataatgttaaagttagccaggtgtagtggtgagtgcctgtggtcccacctactggggagactgaggcaagaggatcctttgagcccaggaggtggaggctgtagagagccatgcttgggccacttgcactccagcctgggcaacagagcaagaccctatctctaaaaaaaaaatggaattgaggatgtgtcctctgggtgtgggctctaccacccaccagcctccctctcctgtgggtgctgcctgccacccaccgctgaggtccctgcagcatcaaggtgcccaaacgaagacactctccagctctgagccaggcacccatacagagcccggcagacgcctctgctgctgcagttcttagaatccagagcgcgtggggaatgtgaatttgtgctgctggagccaaacatcccacctccaggttttagctggaggaatccatgtggatatgcaagcaatgtagttataatgaatagcaattgtgggctgggcgcagtggctcatacctgtaatcccagcactttgggaggctgaggtgggtggatcacctgaggtcaggagtttgagaccggcctggccacataatgaaactccgtctctactaaaaatacaaaaaaattagccgggcatagtggcgggtgcctgtaatcccagctactcaggaggctgaggcaggagaatcacttgaaacccaggaggcagaggtttcagtgaggcgagatcgtgccattgcactccagcccgggagacacagcgagactctctttctcaaaaaaagaatagcagctttgctatagcttaggggagcaaaacccgaaagccaccttaagtttcccgaagatagagatccctggggtcctattctgagacagagtcttgctctgtcgcccaggctggagtgcagtggcggatctcagctcactgcagcctccgcctcctgggttcaagcaattctcctgcgtcagcctcccgagcagctgggattacaggcgcccggcaccacccccagctaatttttttttttttgagacagagtctcgcattgtcgcccaggctggagtgcagtggcgcgatcttggctcactgcaagctccacctcccgggttcacgccattctcctgcctcagcctcccgagtagcagggactacaggcgcccgccaccgtgcccggctaattttttttttttgtattattaatagagacgggtttcaccgtgttaggatgatctcgatctcctgacctcgtgatccgcctgcctcggcctcccaaagtgctgggattacaggcatgagccaccttgcctggtccttttttttgtatttttagtagagatgaggtttcaccatgttggccaggctggtctaaactcctgacctcatgatccgcccgcctcggcctcccaaagtgctgggattacaggtgtgggccactgtgcccggccagtcccattctactttttagggacatggaatcatgtaaagatgccatacagaaaacagtatgcaaatccagagacagaggacatctgtgcatgtttctttctttcttcctttttttttttttttttttttttgagatggattcttgctctgtcgcccaggctggagtgcagtggtgcaatctcagctcactgcaacctctgcctcccaggttcaagtgattctcctgcctcagacttccaagtagctgggattacaggcatgccccaccatgcccagctaatttttgtatttttagtagaggcggggtttcaccatgttggcaaggctggtcttgaactcatgacctcaggtgatccacccgcctcagcttcccaaagtgctgggattacaggcgtgagccgccgtggcaacccgcatgcttcttatgtataagaaaaaaacagctagaaagttgtgggttcactggctgggcacatggtggcttgtgtctgtaacctcagcactttgggaggctgagttaggaggatcatgtgaggccgggagttcaagaccatcctaggcaacatagccagaccctgtctgtaccaaatagaaaaaaaaattaacttgatgtggtggtgtgtgcctgtagtctcagctatttgggaggctgaggtgggaggatcacttgagcccaggaagtcgaggctacagtgagctatgattgtgccactgcactccaacctgggcaacagagcaagaccctgaatcaaaaagaaagagatgtatttgccaacacaggggtgttctgcagtggggtgggaaggggacgtgtaggacttcatagtttccttttttttgttttagagacagggtttaaaaaaaaattttttttttttttttgaggcggaatttcattcttgttgcccaggctggagtgatctcggctcactgccatctccgcctcccaggttgaagtgattctcctgcctcagcctcaggcaattttgtatttttagtagagacggggtgactccatgttggtcaggctggtctcaaactcccgacctcgggtgatctgcctgccttggcctcccaaactgctgggattacaggtgtgagccactgtgcctggccccttttttttttttttaagtagggtcttgctaagttgtccaggctggtcttgaactcctggtctcaaatgatcctcctgacttggcctcccaaaaggctgggattgcaggagatatgagccaccgcacccggcccttttataagttttctaaaggaaaggaacatgtaccatcgtgttttattttaattttacatagaagcaggaacaatagcatgaacccccagacacggccccccaagatgcgctgttccgctgtgctgcagccccatttgtccttttctataccatttttggttgtatttgaagcaaatccctgacatcagggcatttcatcccaaaatacttccatctgtgtttctaaaaaaacgaggccattttcttatgtaaccacaacgtgatcaacagtaatcaatgcttaacatctaattctgggccacgttaagaccctcctgattggctcaagaatgtcttttgtgcagttggtttcttgattctttttgttttttttgagacagggtctcactgtgtcacccaggctggagtgcagtggtgcaatcacggctcactgcagcctcagcctcctgcctcagcgatcctcctgcctcagcctcccaagtagctgggaccacaggtttgcaccaccactcccagctaattaaaaaaagtaatttgtagagacgggggtggggtgggggtcttgctatgttgcccaggctggtctcaaactcctagactcaaacaatcctcctgccttggcctcccaaagtgttgcgatcacaggcatgagcccctgtccctggcccctattatttctctaactggggaaagtcatgtgggaacagatgtagcttgccttggcctctgaccggccctgcctgcgttcctgggtgctctctgctgcttctcatgtgtgccactgtggggactcggccgcccaccccaccccgtggtgttaccttgcgtgtgtagttctgtgagctcagggctatggtctgccagaactagggggcgtggggccccagtaccagcccaaggcctcctctctgcaggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagtgagtgcctcgctcccacctgtcaccgcccccaccaccttgcctgggctaccccgcctgaccctgtcccgccatcccccaggaatgccagctgctactttgacatcgagtggcgtgaccggcgcatcacactgagggcgtccaatggcaagtttgtgacctccaagaagaatgggcagctggccgcctcggtggagacagcaggtaacactaaagccccagttccctggagccgtcctggagtcctggagggtctggccatgccgtggtcacttggtagccccagccaaggcctgctctgtgctgggcatccccccggactggccccgcactgtcctaccctggggactgctgtgtgaccccagctcctggccctccctctctggtcaccccagcctccaccccactccctgccaggaggctcactgactcccctctttctgggacaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaaggcaggttctcctgtgggcagctgctgggcagggaacccctcggtcggggctggggtcagtgctgcggggagcgccctctgcatccacactggaccctggcttggctcagggccattccaggccctaaagggacaggtgtctgatggccaccagggggctctgggatgcaagcagcccctttccctcttgtctgtgtggttggggggacttaccttgcccacctgacagagaggtgtgtggaggggagagcagggagggaaggagaccaggaaagggagggaggagagcaggggaggggagagccgggaagagaggagagcaggggtggggaggtttctggaaagggtgtgcaaggggaggacgcgcctcggttatgggactggagccccttcccaggaggacccccaacaatccagaggtgcctgttaggattcagaacatggtttttttgtttgtttttttgagactcactccctcaccctggctggacttgcagtggcgttatctcggctcactgcaacctctgcctcctgggttcaagtgattctcctgcctcagcctcccaagtagctgggattacaggtgtgcaccaccacgcctggctaatttttatatttttaaaatttattatttatttattttgagacccagtctggagtgcagtggcgttatctcggctctctgcaacctctgcctcctgggttcaagcgattctcctgcctcagcctcccgagtagctgggactatgtgtgggagccaccatgcctggctaatttttttgtatttttcatagagacgggtttcaccatgttgtccaggctggtcttgaattcgtggcctcaagtgatccgcccacctcagcctcccacagtgctgggtttataggtgtgagccaccacacccggctaattgttttgtatttttagtagagacggagcttcactatgttggcaaggctggctcgaactcctgacctcaagtgatccgcccacctcagcctcccaaagtgctgggattacaggcgtgagccactgcggccgagcagaacacgttctaggacccttgttcatgtgtccatcatggacaggaggacgtgcgggccatagggaccctggctcattccggagccgggactggagggtggggcgtcacccttgggaacacccgtgcccaccctccgctgcccagggtaggggtggggagccaggctttgggccccacttgataaagtcccctccccagactccacaggcaaatactggacggtgggcagtgactccgcggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctgagccttatttctctggaagcggctaagggacggttgggggctgggaccctgggcgtgtagtgtaactggaatcttttgcctctcccagccacctcctcccagccccccaggagagctgggcacatgtcccaagcctgtcagtggccctccctggtgcactgtccccgaaacccctgcttgggaagggaagctgtcgggtgggctaggactgacccttgtggtgtttttttgggtggtggctggaaacagcccctctcccacgtggcagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggcccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcagccccaggccggcctgtgtgtgtcttggggctgaggtgggtgggggggctgaggtgggtgggagggctggcgggacaggtaggcgccctggctccccagctcagtgctgggagtgtgcagtgggagggaggccgtggctccagtgggtgctccggagctc

(19) United States
(12) Patent Application Publication
(10) Pub. No.: US 2004/0132674 A1
(43) Pub. Date: Jul. 8, 2004

(54) Regulatory sequence for the specific expression in dendritic cells and uses thereof

(76) Inventors: Angelika Reske-Kunz, Essenheim (DE); Xiaolan Ross, Mainz (DE); Ralf Ross, Mainz (DE); Matthias Bros, Mainz (DE)

Correspondence Address: FISH & NEAVE, 1251 AVENUE OF THE AMERICAS, 50TH FLOOR, NEW YORK, NY, 10020-1105, US

(21) Appl. No.: 10/181,174

(22) PCT Filed: Jan. 12, 2001
(86) PCT No.: PCT/EP01/00362

(30) Foreign Application Priority Data
Jan. 13, 2000 (DE) 100-01-169.1
Mar. 2, 2000 (DE) 100-10-188.7

(51) Int. Cl. A61K 48/00; C07H 21/04

(52) U.S. Cl. 514/44; 536/23.2

(57) ABSTRACT

The present invention relates to regulatory sequences which mediate dendritic cell-specific expression. The regulatory sequences are isolated from the human fascin gene and also comprise for instance promoter sequences. Moreover, described are recombinant nucleic acid molecules and vectors which contain the regulatory sequences and as preferred embodiments, recombinant nucleic acid molecules and vectors encoding antigens or immunoregulatory proteins. Furthermore, the invention relates to host cells, which contain the recombinant nucleic acid molecules or vectors, and to methods for their preparation. Other embodiments relate to in vitro methods for stimulating T-cells and for preparing T cell-stimulating dendritic cells and for formulating them as pharmaceutical compositions. Additional pharmaceutical compositions are described which essentially relate to DNA vaccines and gene therapeutic pharmaceutical compositions, for instance for the immunization against and treatment of infectious diseases, tumors, allergies, Creutzfeldt-Jakob plaques or Alzheimer plaques. Additional pharmaceutical compositions according to the invention can be used for targeted, dendritic cell-mediated modulation of immune responses, for instance to treat autoimmune diseases or graft rejection. Moreover, various uses of the regulatory sequences are described.

The present invention relates to regulatory sequences which mediate a specific expression in dendritic cells, recombinant nucleic acid molecules, vectors, host cells and methods for preparing the host cells. Moreover, the invention relates to uses of the regulatory sequences and pharmaceutical compositions.

Dendritic cells (DC) being antigen-presenting cells (APC) play a key role in the mobilization of the specific immune defense. Dendritic cells are the only cells capable of efficiently activating so-called naive T-lymphocytes which are at rest and ready for defense. In this process, they are able to induce both CD4⁺ T helper cells and CD8⁺ cytotoxic T cells. Dendritic cells therefore control both the humoral antibody-dominated immune response and the cellular immune response. Dendritic cells are indispensable for efficient immune defense against bacterial, viral and parasitic pathogens and tumor cells. Dendritic cells are also causal to pathological disorders of the immune system, such as autoimmune diseases and allergies. Hence, it is a main objective of medicine to put the functions of dendritic cells to therapeutic use.

Dendritic cells in different stages of differentiation have different functional competence. Three discrete maturation phases can be defined from a functional point of view. 1.) Young immature dendritic cells have a monitoring function. Young dendritic cells are positioned as sentinel cells at strategic points in nearly all organs of the body, and in particular in the epithelia delimiting the body towards the outside. They are involved in taking up foreign substances (antigens) in a soluble or particulate form, antigen processing and peptide loading of MHC molecules. 2.) In the subsequent migratory phase, the dendritic cells migrate as peptide transporters into the draining lymph nodes, in order to settle there in the T-cell areas. 3) In this area, the dendritic cells display their immunogenic potential. The dendritic cells present the processed peptide together with MHC molecules to T-lymphocytes having corresponding receptor specificity. The T-lymphocytes are naïve T-lymphocytes, that is to say, cells which have not yet come into contact with the antigen, and hence show a high activation threshold. The direct cell-cell interaction between the dendritic cells and the T-lymphocytes results in the activation, expansion and thus in the functional recruiting of the peptide-specific T-lymphocytes.

Because of their central function in the mediation of the immune responses, dendritic cells are an essential point of attack for protective vaccinations, an aspect which is to be discussed in more detail hereinafter.

Classic protective vaccination which is above all based on the administration of attenuated pathogens is associated with numerous drawbacks. There are, for instance, the potential risks of an infection if the pathogen should regain its human pathogenity (McKee, Am.J.Trop. Med.Hyg. 36 (1987), 435-442), problems relating to the production and storage of the vaccine (Rabinovich, Science 265 (1994), 1401; Fynan, Proc. Natl. Acad. Sci. USA 90 (1993), 11478) and a possible obstruction of antigen presentation by immunomodulatory properties of the pathogen (Levine et al., in New Generation Vaccine, Woodrow, G. and Levine, M. M., eds., Marcel Decker, New York, (1990), 269-287). In light of this, it becomes increasingly evident that the technique of DNA vaccination is a highly promising alternative. In DNA vaccination shown for the first time by Ulmer (Science 259 (1993), 1745-1749), “naked” DNA, which elicits a protective immune response is injected into the body or is otherwise applied (for an overview see Lai and Bennet, Crit. Rev. Immunol. 18 (1998), 449-484). The DNA contains sequences which encode an antigen of a pathogen or tumor or an allergen and which are under the control of an ubiquitously functioning promoter. The applied DNA is taken up by cells of the surrounding tissue, the antigen is expressed and presented on the cell surface via MHC proteins.

In the course of the development of DNA vaccination strategies, it became increasingly evident that targeted addressing of dendritic cells could offer very promising advantages. This can, for instance, be derived from immunization experiments in which dendritic cells were loaded in vitro with antigens and re-injected (WO 94/02156). In this way, for instance murine dendritic cells pulsed in vitro with synthetic peptides (Mayordomo et al., Nat. Med. 1 (1995), 1297-1302; Celluzzi et al., J. Exp. Med. (1996), 283-87), with acid-eluted peptides of tumor cells (Zitvogel et al., J. Exp. Med. 183 (1996), 87-97) and even with intact tumor proteins, (Paglia et al., J. Exp. Med. 183 (1996), 317-22) induced in vivo protective responses of cytotoxic T-lymphocytes (CTL) against the tumor. Human dendritic cells cultured from peripheral blood mononuclear cells (PBMC) of healthy donors can also induce CTL responses against tumor antigens (van Elsas et al., Eur. J. Immunol. 26 (1996), 1683-1689; Falo et al., Nature Med. 1 (1995), 649-653) and in vitro cultured dendritic cells have already successfully been used in clinical studies for tumor therapy (Hsu et al., Nature Med. 2 (1996), 540-544). Dendritic cells and cell lines of this cell type which were transfected in vitro with antigen-encoding expression plasmids, can also induce antigen-specific immune responses in vivo after adoptive transfer (Manickan et al., J. Leucocyte Biol. 61 (1997), 125-132; Timares-Lebow et al., J. Invest. Dermatol. 109 (1997), 266; Tüiting et al., Eur. J. Immunol. 27 (1997), 2702-2707). Dendritic cells, into which mRNA from tumor cells has been introduced, have also already successfully been used for vaccination (Boczkowski et al., J. Exp. Med. 184 (1996), 465-72). From these and other findings, the following advantages of DNA vaccination with targeted antigen expression in dendritic cells can be deduced. It is known, for instance, that the presentation of an antigen by non-professional APCs, not providing the necessary co-stimulatory signals for efficient T cell stimulation, leads to poor reactivity or anergy of the T-cells. Restriction of antigen expression to mature dendritic cells might, therefore, lead to a stronger immune response. Moreover, in addition to antigen expression, it might be possible for instance to express additional immunomodulatory proteins in DCs by co-transfection and thereby to exert a regulatory influence on the immune response. Limitation of expression of these mediators (for instance cytokines, co-stimulatory membrane proteins) to transfected dendritic cells would allow the desired immune response to be very specifically influenced, without fear of side effects in the case of systemic administration of these mediators.

In consequence of this, there is a need for promoters or regulatory sequences which mediate a specific expression in dendritic cells. This would allow for instance DNA vaccination constructs to be prepared, which after broadly applied administration, for instance by intramuscular injection or biolistic transfer into the skin, express the antigen or immunomodulatory proteins only in dendritic cells which are present in relatively small numbers in peripheral tissues. There is at least a need for promoters or regulatory sequences which mediate a dendritic cell-specific expression in those tissues, in which the dendritic cells are loaded with antigens. This occurs primarily in the circulatory system, the skin tissue, mucosal tissue and the muscles. Other possible uses of such promoters or regulatory sequences are in vitro transfections of heterogeneous cell populations, the antigen being exclusively expressed in mature dendritic cells.

A promoter with specificity for dendritic cells has already been described in the art (Brocker, J. Exp. Med. 185 (1997), 541-550). This promoter was isolated from the murine CD11c-gene and was capable of mediating specific expression in a transgene (MHC-class II-I-E-protein) in transgenic mice. However, the promoter presented in this publication does not meet in some points the requirements which the promoter sketched out above for specific expression in dendritic cells has to satisfy.

For instance, dendritic cell specificity could be shown only for spleen and thymus tissue. By contrast, this promoter also expressed the transgene in some of the peritoneal macrophages. Moreover, it is known that CD11c is expressed only in a subpopulation of the dendritic cells (Rich et al., Poster No. D6, 5^{th} International Symposium on Dendritic Cells in Fundamental and Clinical Immunology, Pittsburgh, Penn. U.S.A. 23-28 Sep. 1998). However, what is more important is the fact that the promoter activity shown refers to the murine system. As the envisaged promoter is to lend itself to applications in human medicine, the CD11c promoter does not offer a solution to the problem posed, because the promoter cannot be expected to have the same tissue or cell type specificity in humans and in mice. An example showing different cell type specificity between mice and humans is the chemokine gene DC/B-CK. This gene is expressed in mice only in dendritic cells and activated B-cells but not in macrophages (Ross, J. Invest. Dermatol. 113 (1999), 991-998). By contrast, the homologous human gene is expressed in macrophages (Godiska, J. Exp. Med. 185 (1997), 1595).

Moreover, Tubb et al. have already published an about 3.5 kb genomic 5′-flanking sequence of the murine fascin gene bearing the Genebank/EMBL accession number U90355. Although there are some indications that fascin is predominantly expressed in dendritic cells, it is questionable whether this sequence contains a promoter capable of mediating a specific expression of a transgene in dendritic cells. In any case, except for the raw sequence, no information on this is available. In summary, to date there is no promoter available which would mediate a dendritic cell-specific expression in humans.

Hence, there continues to be a need for such promoters or regulatory sequences for targeted immunizations or immunotherapies focused on dendritic cells to be carried out.

Consequently, the technical problem underlying present invention is the provision of a regulatory sequence which mediates a specific expression in dendritic cells in humans.

According to the invention, this problem is solved by the provision of the embodiments characterized in the claims.

Thus, the present invention relates to regulatory sequences selected from the group consisting of

(a) regulatory sequences comprising the nucleotide sequence indicated under SEQ ID NO. 72 from position 1 to 3069 or the nucleotide sequence indicated under SEQ ID NO. 1;

(b) regulatory sequences comprising the nucleotide sequence contained in the insertion of clone DSM13274 and obtainable by amplification using a pair of oligonucleotides, which for instance have the sequences indicated under SEQ ID NOs. 36 and 37;

(c) regulatory sequences comprising a nucleotide sequence of SEQ ID NO. 72 from position 1136 to 3069, 1451 to 3069, 1621 to 3069, 1830 to 3069, 2127 to 3069, 2410 to 3069 or 2700 to 3069 or selected from the group consisting of: SEQ ID Nos. 2 to 8;

(d) regulatory sequences comprising a nucleotide sequence contained in the insertion of clone DSM13274 and obtainable by amplification using a pair of oligonucleotides, the sequences of the oligonucleotides being for instance indicated under SEQ ID numbers, selected from the group of pairs consisting of: 38 and 37; 39 and 37; 40 and 37; 41 and 37;42and37;43 and 37; and44 and 37;

(e) regulatory sequences comprising at least a functional part of a sequence indicated in (a) to (d) and causing dendritic cell-specific expression; and

(f) regulatory sequences comprising a nucleotide sequence hybridizing with a regulatory sequence indicated in (a) to (e) and causing dendritic cell-specific expression.

The regulatory sequences of the present invention impart a dendritic cell-specific expression to nucleotide sequences which are controlled by them.

The term “regulatory sequence” refers to nucleotide sequences which influence the expression level of a gene, for instance by rendering expression tissue- or cell specific. In this sense, regulatory sequences are understood to mean elements hereinafter also called regulatory elements, which impart to a minimal promoter additional expression properties exceeding the basal, constitutive expression characterizing minimal promoters. In the context of the invention, the term “minimal promoter” refers to nucleotide sequences which are necessary to initiate transcription, that is to say to bind RNA polymerase, and for instance contain the TATA box. Moreover, the term “regulatory sequence” also includes sequences outside the 5′-flanking promoter region. Such sequences are functional in both orientations and are less fixed in their position than promoters, they are preferably within the region of the non-translated translated sequences of the human fascin gene as they are disclosed within the framework of the present invention (SEQ ID NOs. 1 to 20 or the non-translated sequences of SEQ ID NO. 72). Such sequence elements include enhancers and silencers which may regulate the expression of a gene up or down. Enhancers or silencers are often located in introns or in the 3′-flanking region of a gene. The regulatory sequence may also be a promoter which within the meaning of the invention is characterized by exerting all functions of a promoter, that is to say initiation of RNA polymerization, mediation of a specific expression strength and regulation of expression, preferably depending on the cell type, especially preferably with specificity for dendritic cells. The sequences represented in SEQ ID NOs. 1 to 8 or the above-mentioned segments of SEQ ID NO. 72 are regulatory sequences which at the same time correspond to the definition of a promoter.

In the context of the present invention “dendritic cell-specific expression” means expression exclusively in dendritic cells, if the cells are cells of the skin tissue, preferably the epidermis, the mucosal tissue, the lymphatic and blood system and the muscles, preferably if such cells are transfected with an expression construct containing a regulatory sequence according to the invention. In the case of blood cells from persons infected with the Epstein-Barr Virus (EBV), dendritic cell-specific expression additionally refers to B lymphocytes. The regulatory sequences according to the invention may however very well mediate an expression also in cells of other types of tissue, such as neurons, glia cells or fibroblasts, if they ensure that, in the skin, preferably the epidermis, the mucosal tissue, blood and muscle expression specifically occurs only in dendritic cells. The regulatory sequences of the invention are preferably applied in such a way that expression is ensured to take place exclusively in dendritic cells. This is the case for instance with the skin, preferably the epidermis, the mucosal tissue, the blood or muscle.

“Dendritic cells” are antigen-presenting cells which are the only ones capable of efficiently activating naïve T-cells. Dendritic cells also include the Langerhans cells of the skin tissue, which represent immature dendritic cells.

The specificity of the regulatory sequences of the invention is apparent from the expression behavior of the fascin gene from which the regulatory sequences have been derived. No fascin expression has been detected for instance in the skin, preferably the epidermis, nor in the Langerhans cells, as these cells are immature in the skin. Fascin expression was, however, detected in advanced maturation stages of the Langerhans cells (Ross, J. Immunol. 160 (1998), 3776-3782). Fascin expression exclusively in dendritic cells can be shown in blood cells, at least of persons not infected with the Epstein-Barr Virus (EBV) (Mosialos, Am. J. Pathol. 148 (1996), 593-600). Fascin expression was additionally detected in EBV-transfected B-lymphocytes (Mosialos, J. Virol. 68 (1994), 7320). Very low fascin expression which might be attributable to the presence of dendritic cells can be demonstrated in the muscle on the RNA level. (Mosialos, J. Virol. 68 (1994), 7320).

The regulatory sequences of the invention are disclosed by the nucleotide sequences indicted under SEQ ID NOs. 1 to 8 or the above-mentioned corresponding segments of SEQ ID NO. 72.

The regulatory sequences of the invention were provided by DNA sequencing of the human fascin gene, the complete sequence of which is shown in SEQ ID NO. 72 and in FIG. 9. Preliminary DNA sequencing of said gene resulted in the partial nucleotide sequences indicated under SEQ ID NOs. 10 to 15 and 33 to 35 and in FIG. 2. The regulatory sequences SEQ ID NOs. 1 to 20 disclosed in the invention are taken from said preliminary sequences. Differences between the more recent complete sequence and the older partial sequences should generally be attributable to inaccuracies in preliminary DNA sequencing.

The regulatory sequences are also disclosed by deposited clone DSM13274 and the statement of oligonucleotides by which the respective partial sequences of the insertion can be amplified, for instance by PCR. Clone DSM13274 is the PAC clone RPCIP704C24766Q3/4 which was deposited at the Deutsche Sammlung von Mikroorganismen und Zellkulturen, Braunschweig, under the deposit number DSM13274 on Feb. 1, 2000. With the use of oligonucleotides, for instance those the sequences of which are indicated under SEQ ID NOs. 36 and 37, the complete promoter of the human fascin gene can be amplified. By means of oligonucleotide pairs, for instance those the sequences of which are indicated under SEQ ID NOs. 38 and 37; 39 and 37; 40 and 37; 41 and 37; 42 and 37; 43 and 37; or 44 and 37, 5′-deleted fragments of the fascin promoter (see FIG. 4), which allow dendritic cell-specific expression, can be amplified. Additional oligonucleotide pairs which enable a skilled person to amplify the corresponding promoter fragments from the deposited clone can be derived from the nucleotide sequences indicated in SEQ ID NOs. 1 to 8 and 72.

When the regulatory sequences of the invention are provided from the fascin promoter by amplification from clone DSM13274, the specificity of the PCR reaction can be increased by a preceding additional PCR reaction. In such a “nested PCR”, a larger region of the fascin gene locus which contains the desired regulatory sequences is amplified first, for instance using oligonucleotides, the sequences of which are indicated in SEQ ID NOs. 69 and 70. The product of the first PCR reaction can then serve as a template for a second PCR reaction in which one of the regulatory sequences of the invention can be obtained by means of one of the above-mentioned oligonucleotide pairs.

Moreover, the sequence of the promoter fragments can be detected by direct sequencing with the deposited clone serving as a template. To this end, a skilled person can derive sequencing primers from the nucleotide sequences indicated under SEQ ID NOs. 1 to 8 and 72.

The invention is based on the finding that an about 3.0 kb promoter fragment of the human fascin gene (pFascin-3.0, FIG. 4, SEQ ID NO. 1 and position 1 to 3069 in SEQ ID NO 72, respectively) cloned upstream of the coding region of a reporter gene (Photinus luciferase) led to the expression of the reporter gene in transfected, cultured dendritic cells (see Example 2). Moreover, it was possible to show that this expression is specific for dendritic cells since expression in THP-1-cells (human monocyte cell line which does not express fascin endogenously, see FIG. 5) was about 8 times lower. Further expression studies showed that the fascin promoter pFascin-3.0 and a sub-fragment thereof (pFascin-1.6) are able to mediate specificity for mature dendritic cells (CD83⁺ fraction) (Example 2 and FIG. 7). The strength of the fascin promoter was determined by co-transfection experiments using a reporter gene construct containing the unspecific, strongly expressing promoter of the housekeeping gene EF1α (Example 2 and FIG. 8). It is noteworthy that the fascin promoter exceeded the gene expression of the EF1α construct in mature dendritic cells by about one and a half times.

For a further functional characterization of the promoter, 5′-deletions of the 3.0 kb fragment were produced and also functionally analyzed in reporter gene assays (see Example 2, FIGS. 4 and 6). Surprisingly, these analyses showed that a 211 bp promoter fragment (pFascin-01 1, SEQ ID NO. 21 and position 2859 to 3069 in SEQ ID NO. 72, respectively) showed the same expression strength in both cell types examined. By contrast, a fragment further deleted by 56 bp (pFascin 0.05, SEQ ID NO. 22 and position 2915 to 3069 in SEQ ID NO. 72, respectively) still containing the TATA box did not show any expression exceeding the negative control to any degree to speak of. Consequently, pFascin-0.11 is a minimal promoter imparting basic transcription, not exerting dendritic cell-specificity vis-à-vis monocytes.

Surprisingly, it has been found that apparently in more distal regions there are elements which on the one hand increase transcription in dendritic cells (3.3 times as a maximum compared to the minimal promoter) and on the other hand there are elements lowering transcription in THP-1-cells (3 times as a maximum). The promoter construct pFascin-1.4 (SEQ ID NO. 4 and position 1621 to 3069 in SEQ ID NO. 72, respectively) showed the highest specificity with an about 10-fold higher expression in dendritic cells than in THP-1-cells. These findings allow the conclusion that the promoter sequence contains regulatory elements capable of conferring cell type-specificity upon a minimal promoter, as for instance the promoter fragment contained in pFascin-0.11.

Thus, the invention also relates to functional parts of the promoter sequences SEQ ID NOs. 1 to 8 and position 1 to 3069 in SEQ ID NO. 72, respectively, which, for instance in combination with a minimal promoter, mediate dendritic cell-specific expression. Examples of minimal promoters are the SV40 or thymidine kinase minimal promoter.

Moreover, the regulatory sequences according to the invention which represent functional parts of the fascin promoter may be used to modify the expression behavior of existing heterologous promoters. For instance, to a promoter which constitutively expresses a nucleotide sequence controlled by it or which possesses a particular specificity, as for instance inducibility or development specificity, can be imparted an additional specificity for dendritic cells by integration of one or more regulatory sequences of the invention.

It has already been known that the protein fascin which cross-links actin filaments, and thus participates in the formation of the dendrite structure of dendritic cells, is highly expressed by dendritic cells both of man (Ross, J. Invest. Dermatol. 115 (2000), 658-663) and of mice (Ross, J. Immunol. 160 (1998), 3776-3782). Moreover, it has been known that epidermal cells of the non-treated skin (at least in mice) do not show any expression. Only after activation, the mature Langerhans cells, the dendritic cells of the epidermis, express fascin. It has also been shown that fascin is not expressed in human blood cells either, except in dendritic cells (Mosialos, Am. J. Pathol. 148 (1996), 593-600). So far, it has not been possible to provide the sequences which bring about the above-described specificity of fascin expression.

The specificity for dendritic cells of these functional parts can be proved inter alia by the method described in Example 2. Isolation of partial sequences from one of the above-described promoter sequences can be achieved by standard molecular-biological methods known to a skilled person, for instance according to Sambrook et al. (Molecular Cloning: A Laboratory Manual, second edition, Cold Spring Harbor Laboratory Press, Cold Spring Harbor N.Y. (1989)). This source can also be drawn on for all other molecular-biological techniques mentioned in the present description. In order to test the isolated fragments for dendritic cell-specificity, the method described in Example 2 can for instance be used. For this purpose, the fragments upstream of the minimal promoter, as defined above, are cloned and the expression of a reporter gene in dendritic cells and in cells not expressing fascin (for instance THP-1) is subsequently measured in transient assays. Specific expression in dendritic cells within the meaning of the invention is acknowledged if the level of expression compared to the cells not expressing fascin is increased at least 5-fold, preferably at least 8-fold, especially preferably at least 10-fold, particularly preferably at least 15-fold and most preferably at least 20-fold.

Another aspect of the invention relates to regulatory sequences which hybridize to one of the above-defined regulatory sequences of the invention, preferably to the complementary strand thereof, and cause dendritic cell-specific expression of a nucleotide sequence controlled by them.

These hybridizing sequences may be promoters as defined above or regulatory elements imparting dendritic cell-specificity to minimal promoters.

The term “hybridize” as used refers to conventional hybridization conditions, preferably to hybridization conditions at which 5×SSPE, 1% SDS, 1× Denhardts solution is used as a solution and/or hybridization temperatures are between 35° C. and 70° C., preferably 65° C. After hybridization, washing is preferably carried out first with 2×SSC, 1% SDS and subsequently with 0.2×SSC at temperatures between 35° C. and 70° C., preferably at 65° C. (regarding the definition of SSPE, SSC and Denhardts solution see Sambrook et al. loc. cit.). Stringent hybridization conditions as for instance described in Sambrook et al, supra, are particularly preferred. Particularly preferred stringent hybridization conditions are for instance present if hybridization and washing occur at 65° C. as indicated above. Non-stringent hybridization conditions, for instance with hybridization and washing carried out at 45° C. are less preferred and at 35° C. even less.

Such regulatory sequences preferably show a homology, determined by sequence identity, of at least 50%, preferably at least 60%, particularly preferably at least 70%, advantageously at least 80%, preferably at least 90% and especially preferably at least 95% to the sequence indicated under SEQ ID NO. 1 and the sequence from position 1 to 3069 in SEQ ID NO. 72, respectively, preferably over the entire length of the sequences compared. The hybridizing sequences are preferably fragments having a length of at least 500 nucleotides which have an identity of at least 70%, preferably at least 80%, especially preferably at least 90% and particularly preferably at least 95% with the sequence shown under SEQ ID NO. 1 and the sequence from position 1 to 3069 in SEQ ID NO. 72, respectively. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has an for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program DNASIS (version 6.0, Hitachi Software Engineering Co, Ltd., 1984, 1990). For this purpose, the “default” parameter settings (Cut off Score: 16, Ktup: 6) should also be used. The techniques described in Example 2 can for example also be used to determine whether hybridizing sequences mediate dendritic cell-specific expression.

The regulatory sequences of the invention allow a dendritic cell-specific expression of nucleotide sequences which are controlled by them. As the only sub-population of the antigen-presenting cells (APC) capable of efficiently activating naïve T-cells, dendritic cells have a central position in the mediation and modulation of immune responses. The provision of the regulatory sequences of the invention now opens up the possibility of a more targeted use of dendritic cells and of uses to overcome disadvantages of the methods described in the prior art. This essentially refers to the techniques of DNA vaccination and in vitro transformation of dendritic cells, inter alia for the purpose of gene therapy.

It is known that dendritic cells are suitable starting points for DNA vaccinations. Kinetic studies, in which the immunized tissue was removed at different times after immunization, for instance show that directly transfected dendritic cells which migrated to the lymphatic nodes a short time after immunization, are of great importance for successful immunization (Torres et al., J. Immunol. 158 (1997), 4529-4532; Klinman et al, J. Immunol. 160 (1998), 2388-2392). Moreover, dendritic cells which migrated from the transfected tissue after injection of DNA could be shown to carry the antigen in an immunogenic form and to be capable of eliciting immune responses against the plasmid-encoded protein (Casares et al., J. Exp. Med. 186 (1997), 1481-1486).

DNA vaccinations are primarily carried out by injection, for instance by intramuscular or intradermal injection, but can, for instance, also be applied by shooting particles to which the expression plasmids are bound into the skin. Expression plasmids can also be administered orally or sublingually or can be applied to the mucous membrane of the respiratory tract by nasal or intratracheal administration. The induction of humoral or cellular immune responses by application of antigen-encoding DNA into the skin is already being tried out in humans after very good results achieved in animals. So far, promoters have been used which lead to the expression of the encoded protein in all skin cells, and thus for instance in keratinocytes too. Here, however, the epidermal sub-population of the dendritic cells, the Langerhans cells, is the actual addressee of such vaccinations. Experiments with bone-marrow-chimeric mice show that the MHC restriction of the T-cell response after DNA immunization is solely dependent on the MHC haplotype of the bone marrow cells, i.e. on the professional APCs (dendritic cells), and not on the MHC haplotype of the myocytes or keratinocytes (Fu et al., Mol. Med. 3 (1997), 362-371). Consequently, these cells are themselves not able to induce an immune response. Nevertheless, the expression of the antigen in these cells might contribute to immunization mediated by dendritic cells, when the secreted antigen is taken up by dendritic cells. The antigen might then be processed by the dendritic cell and presented via MHC molecules. Such exogenously taken up antigens are presented via MHC class II proteins. However, an antigen presentation via MHC class I-proteins is desirable for an effective DNA vaccination strategy, because this induces the cellular immune defense, while MHC class II presentation elicits a humoral immune response. Contrary to conventional DNA vaccination methods, regulatory sequences of the present invention now allow this to be realized better. In contrast to the uptake of exogenous antigens, intracellular expression of an antigen in dendritic cells leads to the presentation of antigenic peptides via MHC class I-molecules. The targeted addressing of antigen-expressing vectors to dendritic cells largely prevents expression in co-transfected cells in the neighborhood, such as for instance keratinocytes, in a scattered manner, and thus also prevents expressed antigens from being taken up exogenously by dendritic cells via endocytosis. Thus, by vaccination with DNA which is specifically expressed in dendritic cells, a strengthening of the cellular immune response with participation of cytotoxic T-cells can be expected.

Moreover, it is known that the presentation of an antigen by non-professional APCs which do not provide the necessary co-stimulatory signals for efficient T-cell stimulation leads to an insufficient reactivity or anergy of the T-cells. The regulatory sequences according to the invention now allow such effects to be largely eliminated by restriction of the expression of the antigen after DNA vaccination to mature dendritic cells. This leads to an on the whole amplified immune response.

Another important use of the regulatory sequences of the invention resides in DNA vaccination with constructs expressing immunomodulatory proteins (for instance cytokines, co-stimulatory membrane proteins). In addition to the expression of antigens, the immune response can be influenced in a regulatory manner by co-transfection of constructs encoding immunomodulatory proteins. Restriction of these mediators to transfected dendritic cells allows the desired immune response to be very specifically influenced without fear of side effects as are to be expected in the case of systemic administration of the mediators.

Transfection of dendritic cells in vitro, inter alia for subsequent therapeutic applications, can also be decisively improved by the regulatory sequences of the invention. As described above, there already exist numerous prior art methods for using dendritic cells, matured in vitro, for therapeutic purposes which in the meantime already include clinical tests. Many of these methods and applications, however, require the use of as homogenous as possible dendritic cell populations in order to avoid the known disadvantages of transfection of mixed populations, said populations having to be prepared by laborious purification procedures. Dendritic cells are present in the peripheral blood leukocyte population in an amount of only 1% or less. The afore-mentioned drawbacks for instance include the effect of insufficient reactivity or anergy of T-cells as a consequence of antigen presentation by non-professional APCs, discussed above in connection with DNA vaccination.

An example of therapeutic approaches with in-vitro transfected dendritic cells refers to the expression of tumor- or pathogen-derived antigens in dendritic cells which can be used for subsequent therapy in patients.

Another example of a therapeutic form by targeted expression in dendritic cells relates to dendritic cells which present a relevant antigen (for instance an autoantigen or graft antigen) and are efficiently transfected with the IL-10 gene or another immunosuppressing gene. This may be used for induction of targeted anergy in corresponding T cells. In this way, for instance autoimmunity or graft rejection can be treated.

The regulatory sequences of the invention also allow genes of regulatorily acting proteins (for instance cytokines, co-stimulatory molecules) to be specifically expressed in dendritic cells and thus the quality of the immune response to be influenced by selection of the respective class of T-helper cells or cytotoxic T-cells (Th1/Th2, Tc1/Tc2) as well as the strength of the immune response.

The advantage of the regulatory sequences of the invention for instance vis-à-vis the constitutively expressing CMV promoter resides in that the entire leukocyte population (in EBV negative persons) or the B-cell depleted leukocyte population (in EBV infected persons) or enriched freshly isolated or cultured dendritic cells can be used in suitable maturation stages, and thus the time-consuming and costly purification of dendritic cells can be circumvented.

In another embodiment of the invention, the above-described regulatory sequences are combined with at least one nucleotide sequence,

(a) which is selected from the group consisting of: the segment of SEQ ID NO. 72 from position 3911 to 13398, 13556 to 13637, 13760 to 14004, 14173 to 15414 and 16791 to 16951 and SEQ ID Nos. 9 to 20, or parts thereof, or

(6) which is contained in the insertion of clone DSM13274 and is obtainable by amplification using a pair of oligonucleotides, the sequences of the oligonucleotides for instance being indicated under the SEQ ID numbers selected from the group of pairs consisting of 45 and 46; 47 and 48; 49 and 50; 51 and 52; 53 and 54; 55 and 56; 57 and 58; 59 and 60; 61 and 62; 63 and 64; 65 and 66; 67 and 68; and 45 and 60.

According to the results of Example 2, reporter expression in THP-1 is distinctly reduced by the tested regulatory sequences compared to basal expression, it is above all about 10 times, as a maximum, lower than in dendritic cells. In light of the fact that no endogenous fascin expression can be shown in THP-1 (FIG. 5), the question arises why expression in THP-1 in transient expression assays is not by far lower. Obviously, control of the fascin promoter is by itself not sufficient to completely repress fascin expression. Hence, it is to be expected that another controlling element, for instance a silencer, represses transcription in the fascin gene in non-dendritic cells. Silencers are preferably located in introns or in the 3′-gene flanking region. Thus, the invention also comprises regulatory sequences which beside the above-described sequences, are additionally combined with one or more of the following sequences: intron sequences of the fascin gene (Segments of SEQ ID NO. 72 from position 3911 to 13398, 13556 to 13637, 13760 to 14004, and 14173 to 15414 and SEQ. ID NOs. 9 to 19, respectively), the 161 bp 3′-gene flanking region (positions 16791 to 16951 in SQ ID NO. 72 or SEQ ID NO. 20) and, each, parts thereof. The regulatory sequences characterized in this embodiment preferably impart to nucleotide sequences which are controlled by them an expression which is yet more specific than that of the regulatory sequences not combined with intron sequences and/or 3′-fanking sequences. “Yet more specific” means that expression by combination with regulatory sequences of the present embodiment differs between dendritic cells and cells not expressing fascin by a factor which is greater than that with the same regulatory sequences without a sequence from the intron- and 3′-flanking sequences in SEQ ID NO 72 or SEQ ID NOs. 9 to 20. Said factor is preferably greater than 10, especially preferably greater than 15, particularly preferably greater than 20, most preferably greater than 30. Expression of a nucleotide sequence controlled by these regulatory sequences in cells not expressing fascin is below the detection limit.

In this context, “combined” means that one or more sequences from the intron- and 3′-flanking sequences in SEQ ID NO. 72 and SEQ ID NOs. 9 to 20, respectively, or parts thereof are cloned according to conventional molecular-biological techniques near a sequence shown in SEQ ID NOs 1 to 8 or near the above-mentioned segments of SEQ ID No. 72, near a functional part thereof or near a sequence which hybridizes to one of the afore-mentioned sequences. “Near” means that the sequence(s) from the intron- and 3′-flanking sequences in SEQ ID NO. 72 and SEQ ID NOs 9 to 20, respectively, or parts thereof is(are) cloned directly or at a certain distance to the afore-mentioned regulatory sequences, upstream, downstream or intermittently. Cloning is carried out, however, preferably downstream because, as is known, the afore-mentioned regulatory sequences (promoters or functional parts thereof), require relatively defined distances from the transcription starting point and from the TATA box, respectively, for their way of functioning, that is to say for binding RNA polymerase or transcription factors.

“At a certain distance” means a distance which is suitable to allow silencers or enhancers to exert their function. The wording “part thereof” means a sequence within a sequence shown under SEQ ID NOs 9 to 20 or an intron- or 3′-fanking sequence in SEQ ID NO. 72 which is suitable to exert a silencer or enhancer function. Such sequences can be delimited by an experimental procedure which is analogous to that of Example 2, that is to say by functional analyses of reporter gene constructs. For this purpose, for instance an isolated partial sequence of a sequence from SEQ ID NOs. 9 to 20 or an intron- or 3′-flanking sequence in SEQ ID NO. 72 can be cloned in a functional promoter reporter gene construct, such as pFascin-3.0, preferably directly adjacent the 5′-end of the promoter fragment or in an intron of the reporter gene. This construct, and for comparison purposes, the same construct without this partial sequence, can be subsequently analyzed in a transient expression assay in dendritic cells, preferably mature dendritic cells, and in cells not expressing fascin. If the difference of the expression level between dendritic cells and cells not expressing fascin is greater in the case of the construct with the partial sequence than in the case of the construct without the partial piece, then this partial sequence comprises a functional silencer or enhancer element. Additional techniques for delimiting such elements are available to a skilled person. The embodiment comprises the above-described regulatory sequences which are combined with at least one nucleotide sequence, which can be provided by amplification from the insertion of the deposited clone DSM13274, using for instance PCR, or parts thereof. For amplification, for instance pairs of oligonucleotides can be used, the sequences of which are indicated by the following SEQ ID numbers: 45 and 46; 47 and 48; 49 and 50; 51 and 52; 53 and 54; 55 and 56; 57 and 58; 59 and 60; 61 and 62; 63 and 64; 65 and 66 (intron sequences); and 67 and 68 (3′-gene flanking region). Further oligonucleotide pairs can be derived for this purpose from the nucleotide sequences indicated under SEQ ID NOs. 9 to 20.

Moreover, the sequence of these fragments can be determined by direct sequencing using the deposited clone as a template. For this purpose, a skilled person can derive sequencing primers from the nucleotide sequences indicated under SEQ ID NOs. 9 to 20 or from the intron- or 3′-flanking sequences indicated under SEQ ID NO:.72.

The present embodiment also relates to the combination of the above-defined regulatory sequences with the entire intron 1 of the fascin gene or parts thereof. The nucleotide sequence of intron 1 is contained in the deposited clone DSM13274 and can be provided by amplification, which can be carried out using an oligonucleotide pair with sequences as for instance indicated under SEQ ID NOs. 45 and 60.

Here too, the specificity of PCR reactions serving to provide sequences from clone DSM13274, which in the present embodiment can be combined with one of the above-described regulatory sequences can be increased by a preceding additional PCR reaction. In such a “nested PCR” a larger region of the fascin gene locus containing the desired sequences is amplified first, for instance with the use of oligonucleotides, the sequences of which are indicated under SEQ ID NOs. 71 and 68. The product of the first PCR reaction can then serve as a template for a second PCR reaction, by which using one of the above-described oligonucleotide pairs, an intron- or 3′-gene flanking sequence can be obtained for combination with the above-described regulatory sequences.

The sequences obtained from clone DSM13274 by amplification can be combined with the above-defined regulatory (promoter) sequences in accordance with the instructions given for the intron- and 3′-franking sequences disclosed in the sequence protocol.

In a particularly preferred embodiment, the regulatory sequences are of human origin.

The regulatory sequences of the invention are preferably DNA or RNA molecules, the DNA molecules being preferably genomic DNA.

Another preferred embodiment of the invention relates to recombinant nucleic acid molecules containing the regulatory sequences of the invention.

The term “recombinant nucleic acid molecule” relates to nucleic acid molecules originating from a different genetic context and combined by molecular biological methods. Here, the term “different genetic context” relates to genomes from different species, varieties or individuals or different positions within a genome. Recombinant nucleic acid molecules can contain not only natural sequences but also sequences which, compared to the natural ones are mutated or chemically modified or else, the sequences are altogether newly synthesized sequences.

The recombinant nucleic acid molecules of the invention show one or more of the above-described regulatory sequences in combination with sequences from another genetic context. An example of a recombinant nucleic acid molecule contains one or more regulatory sequences of the invention in combination with a minimal promoter obtained from a gene other than the human fascin gene. Here, the regulatory sequences are regulatory promoter elements which impart a dendritic cell-specific expression to the whole promoter.

Moreover, the recombinant nucleic acid molecules can contain, apart from a promoter containing one or more regulatory sequences of the invention, a polylinker sequence located downstream thereof and comprising one or more restriction sites into which nucleotide sequences can be cloned by methods known to a skilled person, which thus come under the expression control of the promoter. Said polylinker lies preferably in a region which is situated directly behind the transcription starting point defined by the promoter.

Moreover, the recombinant nucleic acid molecule of the invention may contain a transcription termination signal downstream of the polylinker. Examples of suitable termination signals are described in the state of the art. The termination signal can, for instance, be the thymidine kinase polyadenylation signal. The herein-described recombinant nucleic acid molecules which preferably contain a nucleotide sequence to be expressed can be directly employed for uses within the meaning of the invention, such as DNA vaccinations. The recombinant nucleic acid molecules of the invention may, for instance, be multiplied by conventional in-vitro amplifications techniques, for instance PCR. However, they can also conventionally be multiplied in vivo in a vector, and after nucleic acid preparation and subsequent removal from the vector, for instance by restriction cleavage, can be provided for uses requiring for instance linearized expression units.

Recombinant nucleic acid molecules, which preferably contain a nucleotide sequence to be expressed, can also constitute expression units which are often designated expression cassettes which can be easily cloned into different standard vectors and depending on the vector can thus exert different functions.

For applications of the regulatory sequences of the invention in EBV-infected persons, expression in B-cells can be suppressed by the combination of the regulatory sequences with known silencer elements for B cells. On the other hand, it can also be quite advantageous for EBV-infected B-cells to also express the nucleotide sequence controlled by the regulatory sequences since EBV-transfected B-cells constitute good antigen-presenting cells for activated T-cells and can thus produce an amplification the immune response.

Another preferred embodiment of the invention relates to vectors containing the regulatory sequence of the invention or the recombinant nucleic acid molecule of the invention. The term “vector” relates to circular or linear nucleic acid molecules which can autonomously replicate in host cells into which they are introduced. The vectors may contain the above-characterized recombinant nucleic acid molecules in their full length or may contain, apart from the regulatory sequences of the invention, the components described for the recombinant nucleic acid molecules, such as minimal promoter, polylinker and/or termination signal.

The vectors of the invention may be suitable for replication in prokaryotic and/or eukaryotic host cells. They contain a corresponding origin of replication. The vectors are preferably suitable for replication in mammalian cells, particularly preferably in human cells.

The vectors of the invention preferably contain a selection marker. Examples of selection marker genes are known to a skilled person. Selection marker genes which are suitable for selection in eukaryotic host cells are for instance genes for dihydrofolate reductase, G418 or neomycin resistance.

The vectors of the invention are preferably expression vectors for expression in eukaryotic cells. Such vectors can be constructed starting from known expression vectors by replacement of their promoter or the sequences not belonging to a minimal promoter with the regulatory sequences of the invention or by supplementation with regulatory sequences (regulatory elements). Examples of expression vectors which can be modified in this way are pcDV1 (Pharmacia), pRC/CMV, pcDNA1 or pcDNA3 (Invitrogen).

Another preferred embodiment of the invention relates to the above-described recombinant nucleic acid molecules or vectors, which additionally contain a nucleotide sequence to be expressed, wherein expression of the nucleotide sequence is controlled by the regulatory sequence or a promoter containing the regulatory sequence.

The “nucleotide sequences to be expressed” encode either a protein or (poly)peptide or RNA molecules which display their function on the RNA level. Nucleotide sequences encoding a protein, polypeptide or peptide comprise a coding region which is characterized by a start codon (ATG), a sequence of base triplets encoding amino acids and a stop codon (TGA, TAG or TAA) if it concerns DNA. In the case of RNAs, the thymidine (T) is replaced with uracil (U). In the case of degenerated amino acid codons, the base triplets can be adapted in accordance with the codon usage of the target cells, using prior art techniques. Examples of nucleotide sequences which express RNA molecules are antisense RNA or ribozymes.

Moreover, the invention relates to the above-mentioned recombinant nucleic acid molecules or vectors, the nucleotide sequence to be expressed encoding an antigen.

In connection with the present invention, the term “antigen” relates to molecules which are recognized by an organism as being foreign, and which thus elicit a specific immune response. Antigens are naturally endocytosed by antigen-presenting cells (APC) and are presented on the cell surface together with histocompatibility antigens (MHC) of class II. The antigens comprised by the present embodiment are proteins, polypeptides or peptides. Antigens which are expressed by an expression vector in a dendritic cell are presented by MHC-I-proteins. In this process, the synthesized proteins are cleaved by proteasomes into peptides. The peptides are translocated preferably by TAP1/TAP2 complexes into the endoplasmatic reticulum and bind there to MHC class I molecules. However, some of the peptides also bind to MHC class II molecules in unknown ways.

In a preferred embodiment of the recombinant nucleic acid molecules or vectors, the antigens are tumor- or pathogen-specific.

The antigens which are specifically expressed by recombinant nucleic acid molecules or vectors in dendritic cells are preferably proteins of pathogens such as viruses (e.g. HIV), bacteria, fungi or parasites which elicit an immune response in patients. Pathogen-specific antigens, however, also include proteins or (poly)peptides containing at least one antigenic determinant (epitope) of a pathogen.

Tumor-specific antigens are proteins or (poly)peptides of tumor cells which can elicit a specific immune response. These also include (poly)peptides containing at least one epitope of a tumor-specific antigen.

In addition, the antigens may also be proteins of the Alzheimer plaques (or at least an epitope thereof) which causally participate in the Alzheimer disease.

In another embodiment of the recombinant nucleic acid molecules or vectors according to the invention, the antigen is an autoantigen or a transplantation antigen.

The term “autoantigen” relates to antigens present in a patient's own body which for instance cause an autoimmune disease as a consequence of a disorder in the self-recognition or the regulative mechanisms of the immune system by forming auto-antibodies. Suitable auto-antigens may be for instance determined for a given autoimmune disease by identification of auto-antibodies in the patient.

“Transplantation antigens” are histocompatibility antigens (MHC) of classes I and II which are introduced by allogenic grafts into an organism, where they elicit an immune reaction (graft rejection).

The recombinant nucleic acid molecules or vectors of the invention which can specifically express autoantigens or transplantation antigens in cells, can be used in order to inhibit the immune reaction directed against these antigens. For this purpose, for instance dendritic cells which express these antigens can be transfected in vitro with an additional expression vector which expresses an immunoregulatory molecule (e.g. IL-10) which is capable of inhibiting immune reactions. Administration of such transformed dendritic cells allows a targeted anergy of T-cells specific for autoantigens or transplantation antigens to be produced, and thus the pathological immune response to be treated.

In another preferred embodiment of the recombinant nucleic acid molecules or vectors, the antigens are allergens.

In connection with the present embodiment, “allergens” are proteins or (poly)peptides which may elicit an allergic reaction in organisms, or are at least one epitope from such a protein or (poly)peptide.

Dendritic cells which intracellularly express an allergen, can for instance induce an allergen-specific cytotoxic T-cell response, whereupon cytotoxic T-cells can kill allergen-presenting cells. In this way, it is possible to prevent the activation of allergen-specific T-helper cells, and thus activation of IgE antibody-producing B-cells.

Moreover, dendritic cells can be co-transfected with expression vectors expressing an allergen and an immunoregulatory molecule (e.g. IL-10) capable of inhibiting an immune response. Alternatively, dendritic cells can be co-transfected with expression vectors which express an allergen and an antisense RNA, the latter inhibiting the expression of a co-stimulatory molecule (for instance of the B7-family). In both cases, an anergy can thereby be induced in the allergen-specific cells.

In another preferred embodiment of the recombinant nucleic acid molecules or vectors of the invention, the nucleotide sequences to be expressed encode a protein which regulates an immune response.

The specific addressibility of dendritic cells by the regulatory sequences of the invention offers the possibility, in addition to antigen-presentation, to control an immune response, purposefully, by expressing proteins which regulate an immune response in dendritic cells. Recombinant nucleic acids or vectors expressing such proteins can be administered by in vitro transfection of dendritic cells and subsequent incorporation of the cells into a person or by direct administration of expression constructs for these proteins, for instance by injection. Immunoregulatory proteins should preferably be expressed in dendritic cells which are loaded with an antigen, preferably by expressing the antigen itself, for instance by a vector of the invention, in order for the immune response elicited by the antigen to be regulated purposefully.

Examples of immunoregulatory proteins are cytokines which include lymphokines, monokines, interleukines, chemokines, colony-stimulating factors, interferons and transforming growth factors, such as TGF-β. Additional immunoregulatory proteins are co-stimulating molecules. Nucleotide sequences encoding the immunoregulatory proteins have been described in the art and can be taken from the literature.

An embodiment of recombinant nucleic acid molecules or vectors, wherein the protein regulating the immune response is a cytokine or a co-stimulating molecule, is particularly preferred.

“Cytokines” are defined as substances which are produced and secreted by different cell types and which contribute as intercellular mediators to controlling the activity of other cells. Interleukines, interferons, chemokines, colony-stimulating factors and transforming growth factors are above all important for applications offered by the regulatory sequences of the invention.

“Co-stimulating factors” are to be understood as molecules which are expressed in a membrane-bound manner by professional antigen-presenting cells such as the dendritic cells or are secreted by these cells and are required for the efficient activation of T-lymphocytes. Nucleotide sequences encoding cytokines or co-stimulating molecules are known to a skilled person. For instance, amino acid sequences of cytokines are published in “The Cytokine Handbook” (A. W. Thomson, ed. Academic Press, San Diego, Calif., 1998) and amino acid sequences of co-stimulatory molecules in “The Leukocyte Antigen Facts Book” (A. N. Barclay, M. H. Brown, S. K. A. Law, A. J. McKnight, M. G. Tomlinson, P. A. van der Merwe, eds., Academic Press, San Diego, Calif., 1997). Corresponding encoding nucleotide sequences can be taken from publicly accessible data bases. The recombinant nucleic acid molecules and vectors according to the invention and according to this and the subsequent embodiments, are constructed preferably with the use of nucleotide sequences that are of human origin. The choice of suitable cytokines or co-stimulatory molecules for the specific expression in dendritic cells allows the immune response to be either increased or inhibited.

In a particularly preferred embodiment, the recombinant nucleic acid molecules or vectors of the invention express proteins, which regulate an immune response, wherein the regulation is inhibition.

This embodiment of the invention can be used to transfect cells that carry an antigen which elicits an undesired immune response (for instance autoantigens or transplantation antigens) with recombinant nucleic acid molecules or vectors expressing a protein which inhibits the immune response. This can be used to induce a targeted anergy in the corresponding T-cells. The dendritic cells preferably carry the antigen, because of having been transfected with a nucleic acid molecule or vector of the invention, which expresses the antigen. Cytokines with such an inhibiting activity are described in the literature. They include for instance interleukine IL-10 or the transforming growth factor TGF-β.

Thus, a particularly preferred embodiment relates to recombinant nucleic acid molecules or vectors which express protein IL-10 or TGF-β.

In another preferred embodiment, the recombinant nucleic acid molecules or vectors of the invention express proteins which regulate an immune response, wherein the regulation is an increase of the immune response.

A targeted increase of the immune response can be achieved by administration of recombinant nucleic acid molecules or vectors which express suitable cytokines or co-stimulating molecules. Among cytokines, immunostimulating effects have been described for instance for the interleukines IL-2, IL-4, IL-12, IL-15, IL-18, for the interferons IFN-gamma and IFN-alpha, for the chemokines DC-CK1 and MDC and for the granulocyte/monocyte-colony-stimulating factor (GM-CSF). Nucleic acid molecules encoding these factors can be taken from the state of the art, for instance in the above-mentioned sources.

Thus, a particularly preferred embodiment relates to recombinant nucleic acid molecules or vectors expressing the proteins IL-2, IL-4, IL-12, IL-15, IL-18, IFN-gamma, IFN-alpha, DC-CK1, MDC or GM-CSF.

Another particularly preferred embodiment relates to recombinant nucleic acid molecules or vectors which express a co-stimulatory molecule, preferably a member of the B7-family, ICOS ligand or CD40.

Proteins of the B7 family, ICOS ligand and CD40 are co-stimulatory molecules which are suitable to increase the immune response in the above-mentioned sense. Nucleotide sequences encoding these co-stimulatory factors are also described in the literature (see above).

In another particularly preferred embodiment of the recombinant nucleic acid molecules or vectors of the invention, the nucleotide sequence to be expressed encodes an apoptosis-inducing molecule.

The term “apoptosis” designates programmed cell death which can be induced by exogenous signals. The recombinant nucleic acid molecules or vectors of the present embodiment can be used to be transfected into antigen-loaded dendritic cells, in order to cause T-cells which are specific against the antigen to die. In this way, the number of such T-cells can be reduced and an undesired immune reaction, for instance against autoantigens or transplanatation antigens, can be attenuated.

The state of the art describes some proteins which are membrane-bound, but can also be secreted in part and can in closest vicinity tigger the suicidal program in cells. They include for instance the proteins of the TNF superfamily.

Thus, a particularly preferred embodiment of the invention relates to recombinant nucleic acid molecules or vectors which express an apoptosis-inducing molecule, wherein the apoptosis-inducing molecule belongs to the TNF superfamily.

In another particularly preferred embodiment of the recombinant nucleic acid molecules or vectors of the invention, the nucleotide sequence to be expressed is an antisense sequence or expresses a ribozyme.

The antisense sequences and ribozymes are molecules, the expression of which occurs on the RNA level. “Antisense sequences” are sequences which are complementary to an mRNA present in the target cell or a part thereof, the part possibly comprising the coding region, 5′-and/or 3′-non-translated region. Antisense-RNAs, that is to say the transcripts of the antisense sequence, are capable of hybridizing in vivo to the complementary mRNA and thereby to inhibit its translation.

“Ribozymes” are catalytic RNA molecules. In context of the present invention the ribozymes are preferably those which can bind specifically to an mRNA so as to render it inaccessible to successful translation by exerting a catalytic activity, preferably by hydrolytic cleavage. Instructions for selecting suitable antisense sequences and for constructing ribozymes with the desired sequence specificity are described in the literature and can be found for instance in “Antisense: From Technology to Therapy” (Schlingensiepen, R., Brysch, W., Schlingensiepen, K.-H., eds., Blackwell Science Ltd. Oxford, 1997) or Rossi (AIDS Research and Human Retroviruses 8 (1992), 183).

A particularly preferred embodiment relates to the afore-mentioned recombinant nucleic acid molecules or vectors, the antisense sequence or the ribozyme being specific for an mRNA encoding a cytokine or a co-stimulatory molecule.

The recombinant nucleic acid molecules or vectors of the present embodiment can be used for a targeted inhibition of the expression of a cytokine or co-stimulatory molecule in antigen-loaded dendritic cells. For this purpose, antigen-loaded dendritic cells can be transfected in vitro, antigen-loading being preferably carried out by co-transfection with a recombinant nucleic acid molecule or vector of the invention encoding an antigen. Targeted inhibition of a cytokine or co-stimulatory molecule allows the immune response to the antigen to be modulated. Moreover, the recombinant nucleic acid molecules or vectors of the present embodiment can also be applied in vivo, in order to elicit a general, dendritic cell-specific expression. Such a procedure allows a patient's general immune situation to be modulated.

In another particularly preferred embodiment of the recombinant nucleic acid molecules or vectors of the invention the nucleotide sequence encodes a transcription factor.

The transcription factors are preferably those which induce the endogenous expression of cytokines or co-stimulatory molecules in dendritic cells. Expression of a transcription factor can prompt induction of several genes for cytokines or co-stimulatory factors at the same time. However, the present embodiment of the recombinant nucleic acid molecules or vectors can also be used to inhibit the endogenous expression of cytokines or co-stimulatory molecules in dendritic cells. It is known that transcription factors in combination with other (endogenous) transcription factors may possess a repressor activity.

In a particularly preferred embodiment of the above-described recombinant nucleic acid molecules or vectors, the recombinant nucleic acid molecules or vectors contain, apart from the one nucleotide sequence to be expressed, a second nucleotide sequence to be expressed, one nucleotide sequence encoding an antigen as defined above and the second nucleotide sequence encoding a protein which regulates an immune response. The immune response can be regulated in that the second nucleotide sequence expresses a cytokine, co-stimulatory molecule, an apoptosis-inducing molecule, a transcription factor, an antisense sequence or a ribozyme. Here, said two nucleotide sequences can be located behind one another in one reading frame, that is to say, being translationally fused (if both nucleotide sequences encode a protein). These coding regions can be directly adjacent to one another or can be spaced apart by a spacer. A spacer separates the tertiary structure of the two proteins spatially from one another, in order to prevent their tertiary structures from negatively interacting. The spacer has, however, preferably the function of acting as a point of attack for a protease, preferably an endogenous protease of the transfected cell, with the result that the expressed proteins are separated in vivo. Alternatively, the spacer can contain an IRES sequence (internal ribosomal entry site). This allows both genes to be transcribed under the control of a single promoter, their translation occurring separately.

On the other hand, the two nucleotide sequences can also be encoded transcriptionally independently from each other. For this purpose, each nucleotide sequence is under the control of its own promoter, with at least one promoter, preferably both promoters, comprising the regulatory sequences of the present invention.

Such an embodiment would allow the particular advantages of co-transfection with expression constructs encoding an antigen and an immunoregulatory protein, respectively, to be transferred to the in-vivo situation. According to the above-described embodiments such a co-transfection of dendritic cells is only possible in vitro.

Application of recombinant nucleic acid molecules or vectors of the present embodiment makes it possible on the one hand to elicit a targeted antigen presentation by dendritic cells via direct administration to the body, and on the other hand to regulate the induced immune response via the activity of a mediator, with side effects owing to unspecific effects e.g. of the mediator being largely excluded. This embodiment covers all antigens and immunoregulatory proteins which have been defined in the afore-mentioned embodiments.

In a particularly preferred embodiment, the above-described vectors are viruses.

In the state of art, a great number of viral vectors for transfection of mammalian cells ex vivo or in vivo is described. These are always derivatives of mammalian or human pathogenic viruses, which have been deprived of their pathogenic properties by genetic modification. For transfection, viral vectors are packaged in vitro according to methods known to a skilled person, i.e. are provided with viral envelope proteins. DNA and RNA viruses can be used. Examples of viruses for transfection of mammalian, preferably human cells, are Herpes virus, retroviruses, adenoviruses and adeno-associated viruses.

In another embodiment of the invention, the above-described vectors are suitable for gene therapy or DNA vaccination.

Gene therapy and DNA vaccination are based on the introduction of therapeutic or immunizing genes into cells ex vivo or in vivo. Suitable vectors or vector systems and methods for using them for gene therapy or DNA vaccination are described in the literature and are known to a skilled person, see for instance Giordano, Nature Medicine 2 (1996), 534-539; Schaper, Circ. Res. 79 (1996), 911-919; Anderson, Science 256 (1992), 808-813; Isner Lancet 348 (1996), 370-374; Muhlhauser, Circ. Res. 77 (1995), 1077-1086; Wang, Nature Medicine 2 (1996), 714-716; WO 94/29469; WO 97/00957; Schaper, Current Opinion in Biotechnology 7 (1996), 635-640; or Verma, Nature 389 (1997), 239-242 and the references cited therein. The above-described recombinant nucleic acid molecules or vectors can for instance be designed for the direct introduction or for introduction via liposomes or viral vectors, e.g. adenoviral or retroviral vectors.

In another preferred embodiment of the recombinant nucleic acid molecules or vectors of the invention, the nucleotide sequence to be expressed is a reporter gene.

Examples of reporter genes, which allow the expression activity of regulatory sequences, preferably promoters, to be detected, preferably in eukaryotic cells, are described in the literature. Examples of reporter genes encode luciferase, green fluorescent protein, β-galactosidase or chloramphenicol acetyltransferase.

Another embodiment of the present invention relates to a method for preparing genetically modified host cells, characterized in that the host cells are transfected with one of the above-described vectors and the transfected host cell is cultured in a culture medium.

The term “genetically modified” means that the host cell or the host contains, in addition to the natural genome, a nucleic acid molecule or a vector of the present invention, which has been introduced into the host cell or the host or into a precursor. The nucleic acid molecule or the vector can be present in the genetically modified host cell/host either as an independent molecule outside the genome, preferably as a replicable molecule, or may be stably integrated in the genome of the host cell or host.

The introduction of a vector into host cells can be carried out according to known standard methods as for instance described in Sambrook et al. (loc.cit.) Examples of applicable transfection techniques are calcium phosphate transfection, DEAE dextran-mediated transfection, electroporation, transduction, infection, lipofection or biolistic transfer. Subsequent culturing can be carried out using standard methods too, or in the case of the genetic modification of dendritic cells, preferably the methods described in the Examples and the references cited therein.

In another preferred embodiment, the invention relates to host cells which are genetically modified with a regulatory sequence, a recombinant nucleic acid molecule or a vector of the present invention or are obtainable by the above-described method.

The host cell of the present invention can in principle be any prokaryotic or eukaryotic cell and includes inter alia mammalian cells, fungal cells, plant cells, insect cells or bacterial cells. Suitable bacterial cells are those which are generally used for cloning, such as E. coli or Bacillus subtilis.

Examples of fungal cells are yeast cells, preferably those of the genera Saccharomyces or Pichia, particularly preferably of Saccharomyces cerevisiae or Pichia pastoris. Suitable animal cells include for instance insect cells, vertebrate cells, preferably mammalian cells, such as CHO, Hela, NIH3T3, MOLT-4, Jurkat, K562, HepG2 or PC12. Further suitable cell lines are described in the art and can for instance be obtained from the Deutsche Sammlung fur Mikroorganismen und Zellkulturen (DSMZ, Braunschweig).

The embodiment of the host cells which are dendritic cells is particularly preferred.

Dendritic cells are the primary site of application of the regulatory sequences, recombinant nucleic acid molecules or vectors of the invention. Dendritic cells can, for instance, be obtained from peripheral blood leukocytes and Langerhans cells can be obtained from epidermal preparations. The isolated cells can also be precursor cells which can be converted into dendritic cells by suitable in vitro culturing. Corresponding methods are described in the art and can for instance also be found in the Examples and in Ross (J. Invest. Dermatol. 115 (2000), 658-663), Ross (J. Immunol. 160 (1998), 3776-3782) or in references cited therein.

These sources also provide methods for culturing dendritic cells.

Host cells of human origin are particularly preferred in the present invention.

Another preferred embodiment of the invention relates to nucleotide sequences comprising a fragment having a length of at least 15 nucleotides which specifically hybridizes under stringent conditions to a strand of a regulatory sequence of the invention.

Hybridizing nucleotide sequences according to the present embodiment can for instance serve as probes which for instance contribute to identify homologous promoters, preferably regulatory sequences of other genes which, on account of certain corresponding sequence elements, induce an expression pattern comparable to that of the regulatory sequences of the invention. Moreover, these sequences can be used to design suitable oligonucleotides, for instance as PCR primers.

The term “hybridization” has already been defined further above. The nucleotide sequences preferably hybridize under stringent conditions. The fragments have a length of at least 15 nucleotides, preferably of at least 20 nucleotides, particularly preferably of at least 50 nucleotides, especially preferably of at least 100 nucleotides, advantageously of at least 200 nucleotides and most preferably of at least 500 nucleotides.

Another preferred embodiment of the invention relates to a method for the antigen-specific stimulation of T cells in vitro, comprising the steps of

(a) transfecting dendritic cells with a vector containing a nucleotide sequence encoding an antigen, alone or in combination with a vector which contains a nucleotide sequence encoding an immunoregulatory protein, regulation being preferably an increase of the immune response, and the nucleotide sequence preferably expressing a cytokine or a co-stimulatory molecule, an antisense sequence or a ribozyme, or with a vector encoding both an antigen and an immunoregulatory protein;

(b) co-culturing the transfected dendritic cells obtained in step (a) with T-cells; and

(c) detecting the activation of the T-cells of step (b).

The method steps are in principle described in the art and can be found for instance in WO94/02156. The provision of dendritic cells has already been discussed further above. A corresponding method can be found in the appended Examples. T-cells are to be prepared according to prior art methods, as for instance described in “Current Protocols in Immunology” (Coligan, J. E., Kruisbeek, A. M., Margulies, D. H., Shevach, E. M., Strober, W., eds., Greene Publishing Associates and Whiley-Interscience, New York, 1991, vol. 1). The advantage of the method of the invention over the prior art concerns the use of the vectors of the invention. As they permit expression in dendritic cells in a specific manner, it is now possible to process cell populations which require a far lower degree of purification of dendritic cells than required in conventional methods, without having to fear any side effects or risks by transfection of non-dendritic cells.

The T-cells used in step (b) for co-culturing can be naïve or activated T-cells. Detection of activation of the T-cells in step (c) can be carried out according to one or more of the following methods: measurement of proliferation, detection of cytotoxic activity, detection of cytokine production, detection of metabolic activity and detection of activation markers.

Another preferred embodiment relates to a method for preparing a pharmaceutical composition which comprises steps (a) to (c) of the method for the antigen-specific stimulation T-cells in vitro and the additional step of

(d) formulating a pharmaceutical composition by mixing the stimulated T cells obtained in step (c) with a pharmaceutically acceptable carrier.

For formulating cells for administration as a pharmaceutical compostion, the cells are suspended in a pharmaceutically acceptable carrier material. This applies to both the stimulated T cells and the dendritic cells described hereinafter. Examples of carrier material are water, sodium chloride solution, dextrose, glycerole etc. or combinations thereof. In addition, the cell suspension to be administered may contain further substances, such as emulsifying agents, pH buffer, adjuvants or also immunoregulatory factors, such as cytokines. T cells stimulated according to the above-described method can be used for instance to treat serious virus infections as shown for CMV in immuno deficient patients (Walter, New Engl. J. Med. 333 (1995), 1038-1044) or to induce the immune defense against metastases (Nestle, Nature Med. 4 (1998), 328-332).

Another embodiment of the invention relates to a method for the in vitro preparation of the T cell-stimulating dendritic cells comprising the steps of:

(a) transfecting dendritic cells with a vector which contains a nucleotide sequence encoding an antigen, alone or in combination with a vector which contains a nucleotide sequence encoding an immunoregulatory protein, an apoptosis-inducing molecule, preferably belonging to the TNF superfamily, or expressing an antisense sequence or a ribozyme, or with a vector which encodes both an antigen and an immunoregulatory protein; and

(b) culturing the transfected dendritic cells in a suitable medium and/or detecting the T-cell stimulating activity.

Another preferred embodiment relates to a method for preparing a pharmaceutical composition which comprises steps (a) and (b) of the method for the preparation of T cell-stimulating dendritic cells and the additional step of

(c) formulating a pharmaceutical composition by mixing the T cell stimulating dendritic cells obtained in step (b) with a pharmaceutically acceptable carrier.

The dendritic cells obtained by this method can be administered to patients as a pharmaceutical composition to induce targeted immune responses by T-cell activation. The dendritic cells can be injected intradermally, subcutanously, intravenously or, in the case of tumor treatment, into the regions of tumor growth or into the lymph vessels, draining these regions.

In a particularly preferred embodiment of the above-described method, the dendritic cells are of human origin.

Another preferred embodiment of the present invention relates to pharmaceutical composition comprising the recombinant nucleic acid molecules and the vectors of the present invention, the host cells, antigen-specifically stimulated T cells obtainable by to the above-described method, or T cell-stimulating dendritic cells obtainable by the above-described method, and optionally a pharmaceutically acceptable carrier.

In a particularly preferred embodiment of the invention, the pharmaceutical composition is a vaccine.

The regulatory sequences, recombinant nucleic acid molecules, vectors or host cells of the invention can be used to prepare a vaccine, that is to say within the meaning of the invention, a DNA vaccine. Modes of administering DNA vaccines are described in the art, and DNA vaccination has already been successfully used to induce anti-tumor immune responses (Tighe M. et al., Immunology Today 19 (1998), 89-97). Moreover, protective immunity against various forms of diseases has already been achieved by administration of DNA nucleic acid molecules (Fynan, Proc. Natl. Acad. Sci. U.S.A. 90 (1993), 11478-11482; Boyer, Nat. Med. 3 (1997), 526-532; Webster, Vaccine 12 (1994), 1495-1498; Montgomery et al, DNA Cell Biol. 12 (1993), 777-783; Barry, Nature 311 (1995), 632-635; Xu and Liew, Immunology 84 (1995), 173-176; Zhoug, Eur. J. Immunol. 26 (1996), 2749-2757; Luke, J. Inf. Dis. 175 (1997), 91-97; Mor, Biochem. Pharmacology 55 (1998), 1151-1153; Donelly, Annu. Rev. Immun. 15 (1997), 617-648; MacGregor, J. Infect. Dis. 178 (1998), 92-100).

For use as vaccines, the nucleic acid molecules of the invention can be formulated in a neutral form or as a salt. Pharmaceutically effective salts are known to a skilled person. The vaccines of the invention can be used inter alia to treat and/or to prevent infections and are administered in doses which are pharmacologically effective for prophylaxis or treatment.

The vaccination protocols to be used within the meaning of the invention involve active immunization, wherein administration of nucleic acid molecules which specifically express antigens or allergens in the dendritic cells of a person is to induce a protective immune response. The vaccination protocols additionally involve combining antigen expression with immunomodulatory effects by additional administration of nucleic acids which also show a targeted expression of the corresponding mediators in dendritic cells or providing additional supporting medication. Further strategies for obtaining protection given by vaccination involve the administration of in vitro transfected dendritic cells, in vitro activated T cells, in each case according to the above-described methods. Methods and principles can be found in the art and are for instance described by Paul, “Fundamental Immunology” Raven Press, New York (1989) or Morein, “Concepts in Vaccine Development” ed: S. H. E. Kaufmann, Walter de Gruyter, Berlin, New York (1996), 243-264.

Vaccines for injection are typically prepared as a liquid solution or suspension. The preparations can be emulsified or the active ingredient can be encapsulated in liposomes. The active ingredients are often mixed with carrier materials which are compatible with the active ingredient. Examples of carrier materials are water, sodium chloride solution, dextrose, glycerole, ethanol etc or combinations thereof. The vaccine may also contain auxiliary substances, such as emulsifiers, pH buffers and/or adjuvants.

DNA can be administered for vaccination by biolistic transfer instead of by injection (U.S. Pat. No. 5,100,702; Kalkbrenner, Meth. Mol. Biol. 83, 1996, 203-216). For this purpose, DNA, that is to say recombinant nucleic acid molecules or vectors of the present invention, are bound to small particles, for instance gold particles or particles of biocompatible material, and, accelerated by gas pressure, are introduced into the epidermis or dermis. DNA can also be administered orally or sublingually or applied to the mucosa of the respiratory tract by nasal or intratracheal application. (for this, examples are given in Etchart, J. Gen. Virol. 78 (1997), 1577-1580 or McCluskie, Antisense and Nucleic Acid Drug Development 8 (1998), 401-414).

Another preferred embodiment of the invention relates to the use of the recombinant nucleic acid molecules or vectors of the invention which express a nucleotide sequence which preferably encodes an antigen which is particularly preferably tumor- or pathogen-specific or is an allergen, the vector being preferably a virus or suitable for gene therapy or DNA vaccination, alone or in combination with the recombinant nucleic acid molecules or vectors of the invention which express an immunomodulatory protein, preferably being a cytokine or co-stimulatory molecule, its regulation preferably being an increase of the immune response, or which express a transcription factor, as well as to the use of the recombinant nucleic acid molecules or vectors of the invention which encode both an antigen and an immunoregulatory protein, of the host cells of the invention, antigen-specifically stimulated T cells obtainable by the above-described method or T cell-stimulating dendritic cells obtainable by the above-described method for preparing a pharmaceutical composition for vaccination against viruses, bacteria, fungi, parasites, tumors, allergens, Creutzfeldt-Jakob plaques or Alzheimer plaques or for the gene therapy of tumors or viral, bacterial or parasitic infections or of allergies.

Another preferred embodiment of the invention relates to the use of the recombinant nucleic acid molecules or vectors of the invention which express a nucleotide sequence, preferably encoding an antigen, which is particularly preferably an autoantigen, transplantation antigen or allergen, the vector being preferably a virus or suitable for gene therapy or DNA vaccination, alone or in combination with the recombinant nucleic acid molecules or vectors of the invention, which express an immunoregulatory protein, the regulation preferably an being an inhibition, or which express an apoptosis-inducing molecule, a transcription factor, or an antisense sequence or a ribozyme, and to the use of the recombinant nucleic acid molecules or vectors of the invention which encode both an antigen and an immunoregulatory protein, or the use of the host cells of the invention for preparing a pharmaceutical composition for treating autoimmune diseases, graft rejection or allergies.

Another preferred embodiment of the invention relates to the use of the recombinant nucleic acid molecules or vectors of the invention which encode an apoptosis-inducing molecule which preferably belongs to the TNF superfamily, the vector preferably being a virus or lending itself for DNA vaccination or gene therapy, for preparing a pharmaceutical composition for avoiding rejection of grafts or autoimmune reactions. Dendritic cells which are contained in a blood sample of the donor of a graft express the encoded apoptosis-inducing molecule after incorporation of the expression vector. After injection into the recipient of the graft, these dendritic cells cause T cells, which specifically recognize the graft antigens, to die. In autoimmune reactions, autoantigen-loaded dendritic cells which after incorporation of the expression vector express the encoded apoptosis-inducing molecule cause T cells which specifically recognize the autoantigen to die.

Moreover, the present invention relates to the use of the regulatory sequences, the recombinant nucleic acid molecules or vectors of the invention for specifically expressing antigens or immunoregulatory proteins in dendritic cells.

In another preferred embodiment, the present invention relates to the use of the regulatory sequences or the recombinant nucleic acid molecules or vectors of the invention, which preferably express a reporter gene, for identifying and isolating cis-elements from the regulatory sequence which mediate dendritic cell-specific expression.

In another preferred embodiment, the present invention relates to the use of the regulatory sequences or the recombinant nucleic acid molecules or vectors of the invention, which preferably express a reporter gene, for determining the degree of maturation of dendritic cells. This embodiment can be used for instance to determine the degree of maturation of in vitro cultured dendritic cells which are to be used in clinical studies.

Another embodiment of the invention relates to the use of the regulatory sequences or the recombinant nucleic acid molecules or vectors of the invention for identifying and isolating factors which mediate dendritic cell-specific expression.

Moreover, the present invention relates to the use of the regulatory sequences of the invention which comprise a nucleotide sequence from one of the sequences indicated in SEQ ID NOs 1 to 8 or a corresponding promoter sequence from SEQ ID NO. 72 or which comprise a nucleotide sequence contained in the insertion of clone DSM13274 and are obtainable by amplification by using a pair of oligonucleotides, the sequences of which are indicated in one of the following pairs of SEQ ID numbers: 36 and 37; 38 and 37; 39 and 37; 40 and 37; 41 and 37; 42 and 37, 43 and 37; or 44 and 37; parts thereof or sequences which specifically hybridize with the afore-mentioned ones, for blocking transcription factors by the provision of transcription factor binding sites in dendritic cells. As the regulatory sequences of the invention mediate a stage-specific expression in dendritic cells in the gene from which they originate (fascin is not expressed in immature dendritic cells but increasingly in more mature stages), it is possible to inhibit transition from immature dendritic cells to more mature stages by blocking transcription factors which mediate stage specificity. For this purpose, the regulatory sequences of the invention or parts thereof, can be used preferably in the form of oligonucleotides. Inhibition of maturation of the dendritic cells indirectly inhibits primary stimulation of T-cells. This can be used for instance in tissue transplantation to prevent rejection reactions.

These and other embodiments are disclosed and obvious to a skilled person and embraced by the description and the Examples of the present invention. Additional literature regarding one of the above-mentioned methods, means and uses, which can be applied within the meaning of the present invention can be obtained from the prior art, for instance in public libraries, e.g. with the use of electronic means. For this purpose, public data bases, such as “Medline”, can be accessed via the internet, for instance under the address http://www.ncbi.nlm.nih.gov/PubMed/medline.html . Additional data bases and addresses are known to a skilled person and can be taken from the internet, for instance under the address http://www.lycos.com. An overview of sources and information regarding patents or patent applications in biotechnology is given in Berks, TIBTECH 121 (1994), 352-364.

### THE FIGURES SHOW

FIG. 1: The genomic organization of the human fascin gene. Top: Schematic reproduction of the gene locus. The gene extends over about 13 kb and consists of five exons (highlighted as boxes, gray: non-translated, black: protein-encoding regions). Bottom: Position and size of the genomic fragments, subcloned from the PAC clone RPCIP704C24766Q3/4, which have been used for detailed studies. The restriction sites used for the respective subcloning are indicated at the top. H: Hind III, Hi: Hinc II, P: Pst I, S: Sac I, E: Eco RI.

FIG. 2: The nucleotide sequence of the human fascin gene. Exon sequences are indicated in bold letters. The start and stop codons and the putative polyadenylation signal are each twice underlined. Exon-intron splicing sites are written in italics and underlined. (The length of not yet sequenced gene segments is shown in square brackets. For two partial sequences in intron 1, the correct orientation is not yet known because of flanking sequencing gaps).

FIG. 3: The expression strength of the human fascin promoter (pFascin-3.0) in dendritic cells (DC) generated from CD14⁺ precursor cells of peripheral blood compared to the negative control (pGL3-Basic). The normalized expression strength of the tested promoter fragment is indicated as the quotient of the luciferase activities for the test construct pFascin-3.0 (Photinus luciferase) and the constitutively expressed co-reporter pRL-CMV (Renilla luciferase). The means±SEM (standard error) of the constructs tested in triplicates is indicated.

FIG. 4: The position and size of the tested deletion constructs of the human fascin promoter compared to the fascin gene (top). The 5′-gene flanking genomic region (blank section), the transcribed non-translated 5′-gene region (5′-UTR, gray section) and the translated section (black section) of exon 1 and the flanking portion of intron 1 are shown. The putative promoter region was cloned into vector pGL3-Basic in front of the promoter-free reporter gene Photinus luciferase (pFascin-3.0). 5′-shortened deletion constructs were prepared by directed “nested deletion” and relegations, respectively. The respective size of the promoter constructs (minus the 5′-UTR portion) is indicated as the designation of the clone (in Kb).

FIG. 5: Proof of absence of endogenous fascin expression in human monocyte line THP-1 by means of RT-PCR. A: A 266 bp fragment from the 3′-UTR of fascin-cDNA (hFascin-UTR) was amplified. Lane 1: positive control (hFascin-UTR, PCR product cloned into pUC18); 2: negative control (H₂O); 3: SHSY5Y (neuroblastoma line; expresses fascin constitutively); 4:THP-1. B: The cDNA amounts to be used for the fascin PCR shown in A were standardized by HPRT-PCR. Lane 1: THP-1; 2: SHSY5Y; 3: negative control. In A and B, the molecular weight marker (φX174, Hae III restricted) is loaded in each case in the first lane.

FIG. 6: Expression strengths of the deletion constructs of the human fascin promoter in DC and monocyte line THP-1. The normalized values for reporter gene expression of the individual promoter constructs are standardized to the expression strength of the minimal promoter (pFascin-0.11) by the unit one. The means±SEM is indicated for the constructs tested in triplicates.

FIG. 7: Comparison of the activity of the fascin promoter in CD83⁺ and CD83⁻ cells of a DC culture. The relative expression strength of three human fascin promoter constructs of increasing length (pFascin-0.11, pFascin-1.6, pFascin-3.0) were comparatively tested for the CD83-positive and -negative cell fractions of a DC culture. The relative expression strength of each promoter construct is indicated in comparison to the shortest construct with basal activity (pFascin-0.11) the relative luciferase activity of which is standardized to 1. The promoter-free vector pGL3-Basic (pGL3-Basic) served as a negative control. The means±SEM of two experiments, each of which was carried out in triplicates, are indicated. Differences in transfection efficiency were normalized by co-transfection with pRL-CMV.

FIG. 8: Cell type-specific activity of the human fascin promoter. The cell type-specific activity of the human fascin promoter (pFascin-3.0) is indicated relative to the expression strength of a positive control. A luciferase construct was used as a positive control, in which expression of the reporter luciferase occurs under the control of the promoter of the housekeeping gene EF1α, the promoter mediating a strong cell type-independent expression in the tested cells. Both constructs were tested in human cell types: in a fascin-negative keratinocyte line (HaCaT), in a fascin-expressing neuroblastoma line (SHSY-5Y) and in strongly fascin-positive mature dendritic cells (DC). The means±SEM of three (HaCaT, SHSY-5Y) and two (DC) experiments, respectively, each of which was carried out in triplicates, are stated. Differences in transfection efficiency were normalized by co-transfection with pRL-CMV.

FIG. 9: Nucleotide sequence of the human fascin gene. Exon sequences are indicated in bold letters. The start and stop codons and the putative polyadenylation signal are in each case underlined twice. Exon-intron splicing sites are indicated in italics and underlined.

The Following Examples Illustrate the Invention

### Methodic Setup

### Cell Culture and Preparation of Human Dendritic Cells

The human monocyte line THP-1 (Tsuchiya, Int. J. Cancer 26 (1980), 171-176) was cultured in RPMI 1640, the human keratinocyte line HaCaT (Boukamp, J Cell Biol. 106 (1988), 761-771) in IMDM and the human neuroblastoma line SHSY-5Y (Vinores, Cancer Res. 44 (1984),: 2595-2599) in a mixture of DMEM and Nut Mix F12 in equal volumes. The culture media were each supplemented with 10% fetal calf serum (FCS), 2 mM L-glutamine, 1 mM sodium pyruvate, 100 IU/ml penicillin and 100 μg/ml streptomycin. All culture media and additives were purchased from Life Technologies (Eggenstein).

Human dendritic cells (DC) were prepared from peripheral blood mononuclear cells (PBMC) as described in Jonuleit (Eur. J. Immunol. 27 (1997), 3135-3142). All centrifugation steps were carried out at room temperature. About 80 ml of buffy coat obtained from healthy blood donors were diluted 1:3 with phosphate-buffered sodium chloride solution (PBS), containing 2 mM EDTA and 0.4 IU/ml of sodium heparin. 15 ml of Ficoll-Histopaque (Biochrom, Berlin) were overlaid with twice the volume of the diluted buffy coats. After centrifugation at 1000 rpm for 20 minutes a 5 ml aliquot of the upper phase (serum fraction) was removed and heat-inactivated at 56° C. for 30 minutes. The serum fraction was centrifuged at 3000 rpm for 5 minutes and the supernatant was stored as autologous plasma at 4° C. for subsequent uses. The Ficoll gradient was centrifuged a second time (15 minutes at 1500 rpm) and the PBMC-containing interphase was gently removed using a pasteur pipette. The PMBCs were diluted with 30 ml of PBS containing 2 mM EDTA and washed three times with PBS/2 mM EDTA. The cell number was determined and cells were incubated for one hour at 37° C. in X-Vivo 15 (Bio-Whittaker, Walkersville, USA) containing 3% of autologous plasma in 6-well cell culture cluster plates (Corning Costar, Bodenheim) in a volume of 3 ml (5×10⁶ cells/ml). Subsequently, supernatant containing the non-adherent leukocytes was removed and replaced with new culture medium, supplemented with 800 U/ml of recombinant human (rh) GM-CSF and 1000 U/ml of rhIL-4. Cytokine treatment induced maturation of the initially adherent monocytic cells towards immature dendritic cells. Every two days, 1 ml of the medium was replaced with medium supplemented with 1600 U/ml of rhGM-CSF and 1000 U/ml of rhIL-4. On day 7, the non-adherent cells which represent the immature dendritic cells were harvested and washed twice with culture medium. Immature dendritic cells were seeded at a density of 10⁶ cells/ml in 6-well plates in 3 ml of culture medium supplemented with a mixture of cytokines initiating further maturation. The cytokine cocktail consisted of rhGM-CSF (800 U/ml), rhIL-4 (500 U/ml), rhIL-1β (10 ng/ml), rhIL-6 (1000 U/ml), rhTNFα (10 ng/ml) and prostaglandin E2 (1 μg/ml). All cytokines were obtained from Strathmann Biotech (Hannover) except for rhGM-CSF (Sandoz, Nürnberg). Prostaglandin E2 was purchased from Pharmacia & Upjohn (Erlangen). After 24 hours of incubation, mature dendritic cells were used for transfection.

Alternatively, the dendritic cells were separated into a CD83-positive and CD83-negative fraction prior to transfection. For recognition of the DC maturation marker CD83, the murine monoclonal antibody HB15e (BD Biosciences, Heidelberg) was used. The CD83-positive cells of the DC culture were incubated with antibody-coated paramagnetic beads, isolated via magnetic separation, and after DNase-mediated digestion of the beads, they were used for transfections. For cell separation, the CELLection™ Pan Mouse IgG kit (Dynal, Hamburg) was used in accordance with the manufacturer's instructions.

### Reverse-Transcription PCR (RT-PCR)

mRNA was isolated using the QuickPrep™ Micro mRNA Purification Kit (Amersham Pharmacia Biotech, Uppsala, Sweden). The RNA pellet was dissolved in 5 μl of diethyl pyrocarbonate-treated water. The reverse transcription (RT) reaction was carried out with 1 μl of mRNA in a total volume of 20 μl as described by Ross (PCR Methods Applic. 4 (1995), 371-375). Amplification and reverse transcription reactions were carried out using a DNA Thermal Cycler, model 480 (Perkin-Elmer, Forster City, USA). All gene specific primers used for PCR were prepared by MWG-Biotech (Ebersberg). The amount and quality of the cDNA obtained were checked by amplification of a 366 bp fragment of the housekeeping gene hypoxanthine-guanine phosphoribosyl-transferase (HPRT) with 1 μl of the RT reaction as the template. The primers HPRT-3 (5′-GCTGACCTGCTGGATTACAT-3′; SEQ ID NO.23) and HPRT-4 (5′-CATTATAGTCAAGGGCATATCC-3′; SEQ ID NO. 24) were used. PCR denaturation (94° C. for 1 minute) and extension steps (72° C. for 1 minute) were constant. The annealing temperature was decreased from 59° C. to 58° C., each for two cycles, and to 57° C. for another 30 cycles (1 minute each). The final extension step (7 minutes at 72° C.) ensured complete double strand polymerization and was part of each PCR reaction (see also further below). For relative quantification of the amplified HPRT-cDNA fragment, aliquots of the PCR reactions were subjected to gel electrophoresis on 1.4% agarose gel and the fluorescence intensities of the ethidium bromide-stained PCR products were compared using an imaging system (Herolab, Wiesloch). HPRT-standardized amounts of cDNA were used as a template for RT PCR, wherein a 277 bp fragment of the 3′-UTR of human fascin-cDNA (SEQ ID NO. 27) was amplified with the use of the fascin-specific primers hFascin-3 (5′-GGCAAGCCTGGCTGTAGTAG-3′, SEQ ID NO. 25) and hFascin-4 (5′-CCAGAGTGAGATGCATGTTGG-3′; SEQ ID NO. 26). PCR denaturation (94° C.), annealing and extension steps (72° C.) were carried out for 1 minute each. The annealing temperature was decreased from 66° C. to 65° C., each for two cycles, and to 64° C. for another 30 cycles.

### Hybridization Probes

A genomic library was screened using probe mFascin-ORF which encompasses the first two thirds (+73 bp to +984 bp) of the open reading frame (ORF) of murine fascin cDNA (SEQ ID NO. 29). Within this region, murine and human fascin cDNAs show 91% homology on the nucleotide level (GenBank accession Nos. U03057 and L33726). The probe mFascin-ORF was prepared using the primers allfas-1 (5′-GCCACCATGACCGCCAACGG-3′; SEQ ID NO: 31) and allfas-2 (5′-TGTGTGTGTCGCGTCGCGGTCGATCTCCA-3′; SEQ ID NO: 32). Murine cDNA from dendritic cells to be used as template was heat-denatured (98° C. for 5 minutes), and quickly cooled on ice before being used in PCR. During PCR, the denaturation (95° C. for 1 minute) and extension steps (72° C. for 2 minutes) remained constant. The annealing steps were each carried out for 1 minute, and the temperature was decreased from 70° C. to 69° C. and further to 68° C., each for two cycles, respectively, and to 67° C. for another 30 cycles. The probe was labeled during PCR with digoxigenin (DIG)-11-dUTP using the PCR DIG Probe Synthesis Kit (Roche Molecular Biochemicals, Mannheim).

Genomic restriction fragments which contain more distal parts of the gene were identified by probe hFascin-UTR which covers the distal part of the 3′-UTR of the human fascin gene (+2388 to +2664 in SEQ ID NO.27). cDNA from human dendritic cells was employed as the template for amplification using the primers hFascin-3 and hFascin-4 (see above). The PCR product was cloned into Hinc II-restricted pUC 18 and sequenced. Subcloned hFas-UTR was used as the template for the DIG-labeling reaction. The template was heat-denatured at 98° C. for 5 minutes and rapidly cooled.

### Hybridization Conditions

All reagents for filter hybridization with DIG-labeled probes and signal detection as well as the nylon membranes were purchased from Roche Molecular Biochemicals, and the experiments were carried out as reported by Ross (in BioTechniques 26 (1999), 150-155). Briefly, DIG Easy Hyb was used as both prehybridization and hybridization solution and contained 50 mg/ml of sheared and heat-denatured salmon sperm genomic DNA. Both prehybridization and hybridization were carried out at 38° C. in a water bath under mild shaking. The DIG-labeled probe was detected by alkaline phosphatase-coupled anti-DIG antibody and CDP-Star™ as the chemiluminescence substrate. Exposure times for autoradiography were typically less than 30 minutes for arrayed library filters and less than 5 minutes for Southern blots and colony filters. For reuse, the filters were stripped as recommended by the manufacturer.

### Isolation and Characterization of Genomic Clones

The arrayed human genomic library obtained from PBMCs of a Caucasian individual was constructed by P. Ioannou, C. Chen and B. Zhao (Roswell Park Cancer Institute Human Genetics Department, Buffalo, N.Y.) (Ioannou, Nature Genetics 6 (1994), 84-89) and was obtained from the Resource Center of the German Human Genome Project at the Max Planck-Institute for Molecular Genetics (Berlin). The library was screened by hybridization with the probe mFascin-ORF. After DNA preparation (NucleoBond™ Plasmid Kit, Clontech, Palo Alto, USA), positive PAC clones were verified in a Southern blot. For further characterization, PAC clone DNA was cleaved and the resulting fragments were randomly subcloned into pZero2.1 (Invitrogen, Groningen, The Netherlands).

### DNA Sequencing

The nucleotide sequence was determined by cycle sequencing and analyzed on a PE 373A sequencer (Perkin-Elmer). Because of the high GC content of the fascin gene sequences, above all in the 5′-flanking region, which presumably led to the formation of strong secondary structures, some partial regions could not be sequenced according to conventional methods, which could, however, be overcome by the following measures. GC-rich DNA templates were linearized by restriction digestion and heat-denatured (98° C. for 5 minutes) prior to sequencing. Moreover, DMSO was added up to a 5% final concentration. The temperature of the denaturation step was lowered to 95° C., and the number of cycles was increased to 30.

### Construction of a Fascin-Promoter-Photinus-Luciferase-Plasmid

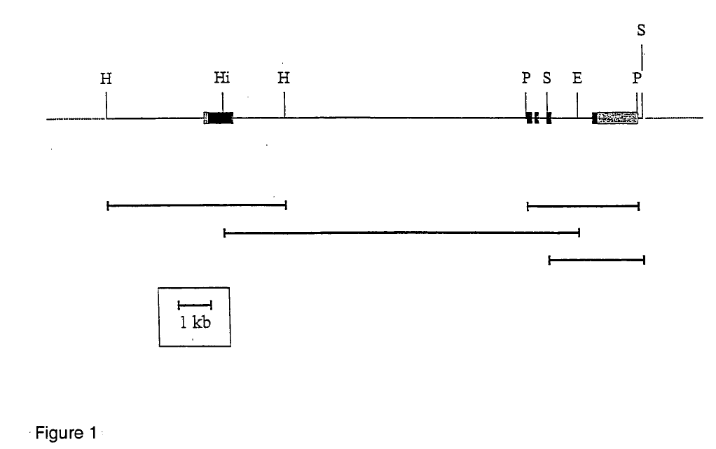

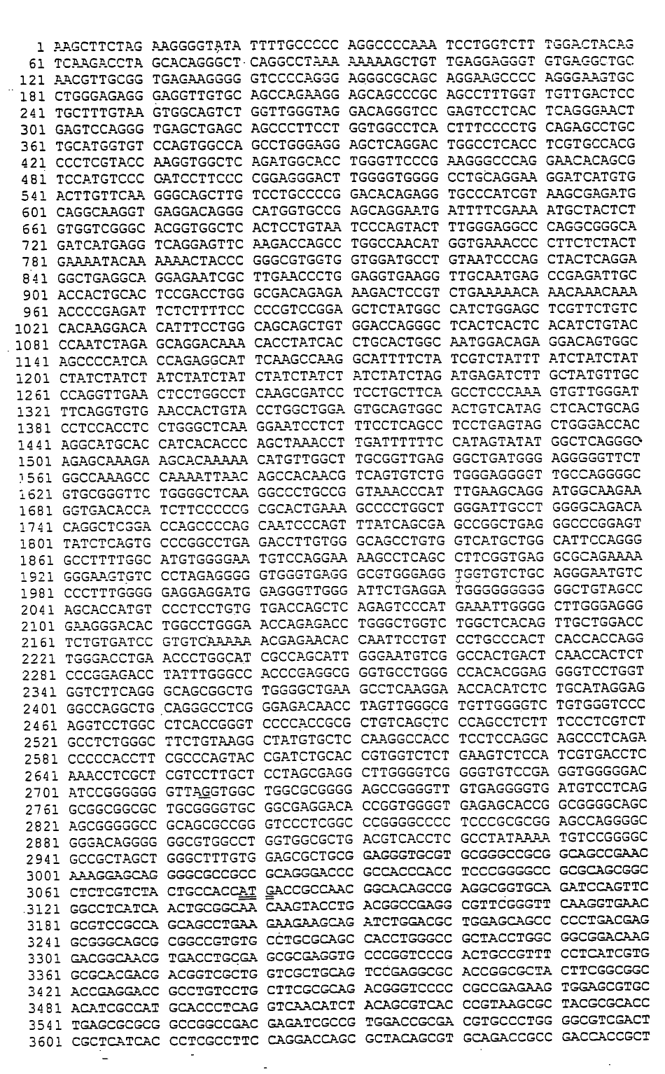

A subcloned genomic Hind III restriction fragment of 5.5 kb contained 3 kb 5′-flanking sequence of the fascin gene, exon 1 and a part of intron 1 (FIG. 1). The putative promoter fragment, encompassing the 5′-flanking gene region and a part of the 5′-UTR was excised (restriction with Acc I, endfilling, and additional digestion with Kpn I) and ligated into the promoter-free Photinus luciferase expression vector pGL3-Basic (Promega, Madison, USA). The correct length and orientation of the promoter construct (pFascin-3.0) were checked by DNA sequencing of both ends (RVprimer3 and GLprimer2, Promega). For 5′-deletion cloning, the construct was cleaved with Kpn I and Stu I and deletion cloning was performed as recommended by the manufacturer (Amersham Pharmacia Biotech). Several deletion clones were generated by restriction using Kpn I and a second restriction enzyme cleaving in pFascin-3.0. The integrity and length of the plasmid DNA for each deletion clone were checked by gel electrophoresis. To normalize absolute Photinus reporter expression values for differences in transfection efficiency, a CMV promoter-controlled Renilla luciferase expression vector (pRL-CMV, Promega) was used as a co-reporter. Plasmid DNA was isolated using the Qiagen™ Plasmid Kit (Qiagen, Hilden). DNA concentration was determined photometrically. The integrity of the plasmid DNA was checked by gel electrophoresis.

For a comparison of the expression strength of the human fascin-promoter in different cell types, the promoter of the housekeeping gene EF1α was used for standardization (Wakabayashi-Ito, J. Biol. Chem. 269 (1994), 29831-29837). This was to prevent any cell type specific differences in the expression of the generally used reference construct under the control of the CMV promoter from distorting the result. For this purpose, the EF1α promoter from the expression construct PEF-BOS-Iacz (Mizushima, Nucleic Acids Res. 18 (1990), 5322) was amplified by PCR and cloned into pGL3-Basic. In parallel thereto, the CMV promoter of the co-reporter construct pRL-CMV was replaced with the EF I c promoter (pRL-EF1α). Transfection experiments in HaCaT, SHSY-5Y and DC, in which both co-reporters were tested in parallel batches showed that the expression strengths of the EF1α and CMV promoter are of the same order of magnitude.

### Transient Cell Transfection

Transfections were carried out by biolistic gene transfer using the helium-driven PDS-1000/He system (Bio-Rad, Hercules, USA) according to Kalkbrenner (Meth. Mol. Biol. 83 (1996), 203-216). Dendritic cells (5×10⁵) and THP-1 cells (106) were co-transfected with 4.5 pmoles of the test construct and 0.5 pmoles of the pRL-CMV. The distance between the macrocarrier holder and the aspired transwell (24 mm diameter, 3 μm pore size, Coming Costar, Bodenheim) was 6 cm. A pressure of 900 psi was chosen on the basis of optimization experiments.

The human cell lines HaCaT and SHSY-5Y were transfected by lipofection. On the day before that, 5×10⁵ cells were seeded per well of a 24-well cell culture plate. For each transfection, 190 nmoles of test construct and 20 nmoles of co-reporter (pRL-CMV) were used. By addition of denatured salmon sperm DNA (Roche Diagnostics, Mannheim), the total DNA amount was standardized to 1 μg. GenePorter (PEQLAB, Erlangen) was used as the transfection reagent. Based on corresponding optimization experiments, transfection of the HaCaT cells was carried out using 7 μl GenePorter/μg of DNA and transfection of SHSY-5Y was carried out using 4μl/μg DNA. Transfection was carried out as recommended by the manufacturer.

### Luciferase Assays

Cell extracts were prepared 24 hours post transfection. The cells were pelleted and washed with 2 ml of PBS. The cell pellets were resuspended in Passive Lysis Buffer (Promega) at a concentration of 10⁶ cells/100 μl and incubated for 15 minutes at room temperature on a rocking platform under mild shaking. The cell lysates were stored at −20° C. Samples were thawed and placed on ice. 10 μl of cell lysate were analyzed for Photinus and Renilla luciferase activity by the Dual luciferase™ reporter assay system as recommended by the manufacturer (Promega) in a Turner luminometer TD-20/20 (Turner Design, Sunnyvale, USA). Luciferase activity was measured for a period of 10 seconds. Absolute values were normalized by dividing the Photinus luciferase activity by the Renilla luciferase activity.

### EXAMPLE 1

### Isolation and Genomic Organization of the human Fascin Gene

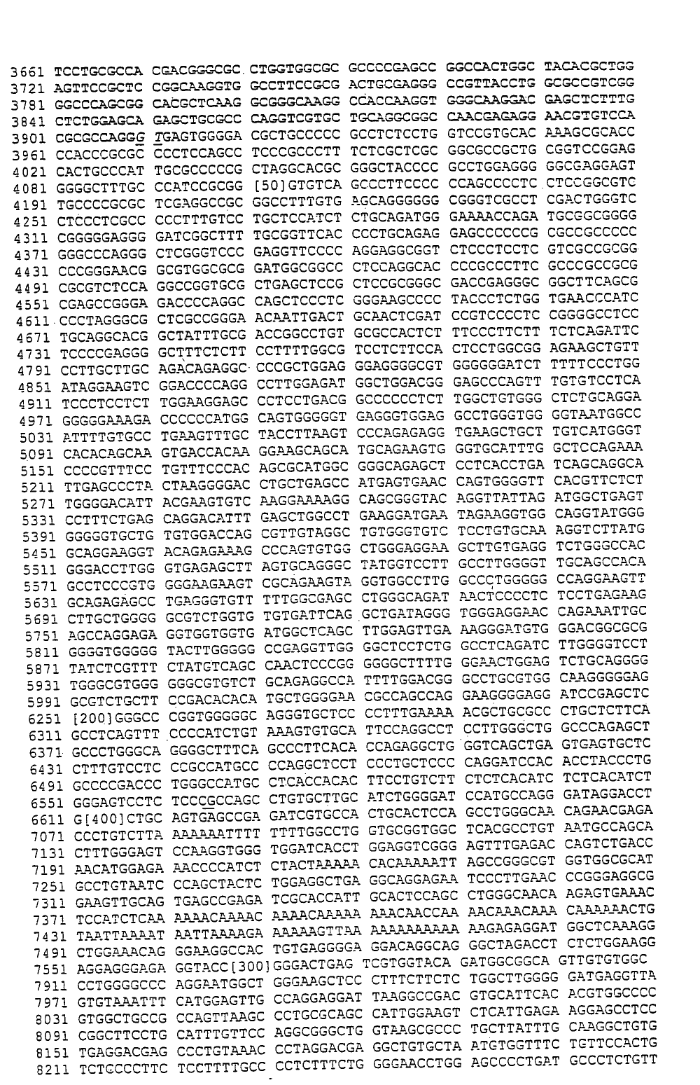

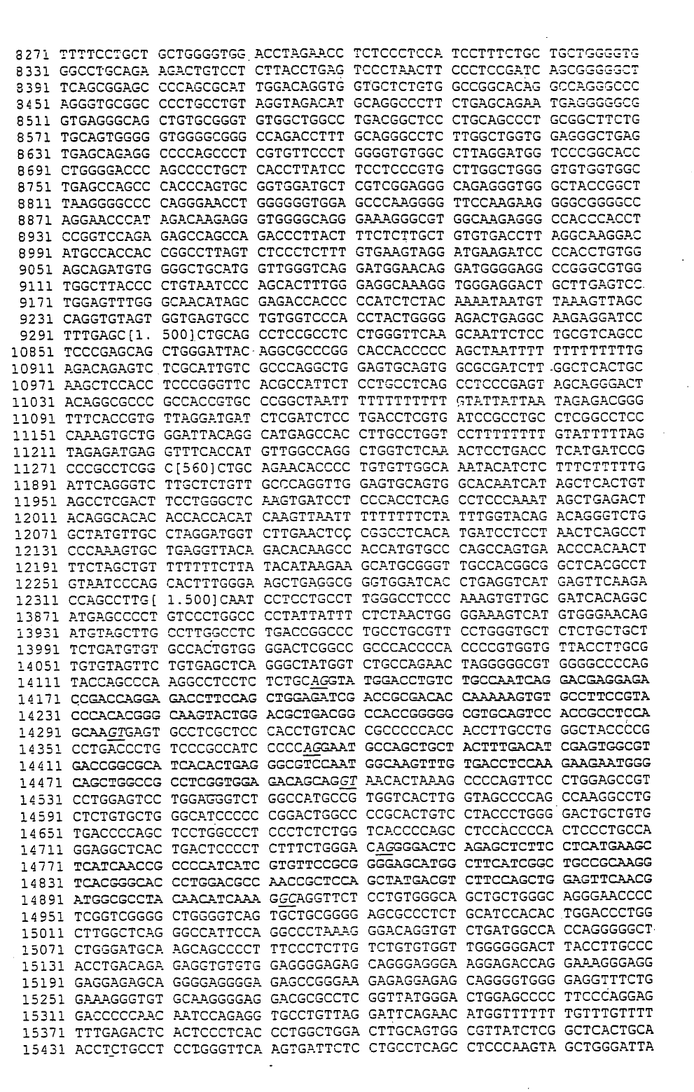

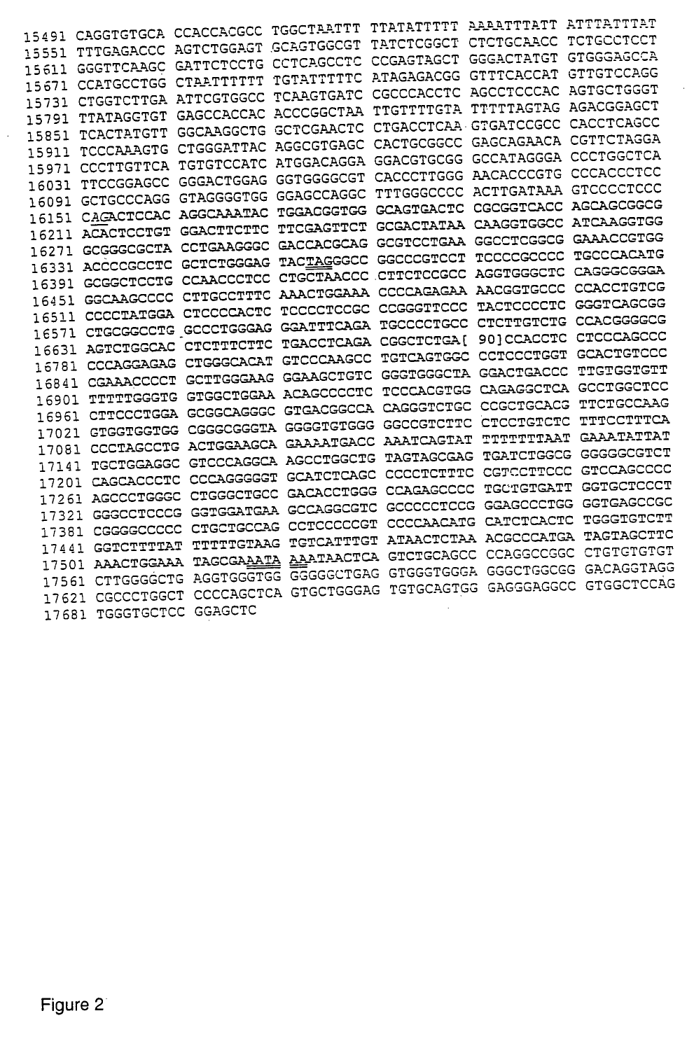

Screening of a human PBMC-derived genomic PAC library with the murine fascin cDNA-specific probe mFascin-ORF identified 16 clones, eight clones of which proved to be positive in a Southern blot. Clone RPCIP704C24766Q3/4 was used to characterize the human fascin gene locus. PAC clone-DNA was digested with Hind III, Pst I, and Sac I, respectively, and restriction fragments were cloned randomly into vector pZero2.1. A larger restriction fragment containing a part of exon 1, complete intron 1 and exons 2-4 was cloned by double digestion with Hinc II and Eco RI. Cloned fragments hybridizing with fascin probes were analyzed further and subjected to sequencing. FIG. 1 shows that the human fascin gene covers a genomic region of approximately 13 kb and consists of five exons. Exon 1 comprises the short 5′-non-translated region (5′-UTR) of the gene and about half of the translated sequence. Intron 1 has a length of about 8 kb. The short exons 2 to 4 are clustered within a region of 800 bp, and encode one third of the translated mRNA. Exon 5 is about 1 kb further downstream and contains the remaining coding region as well as the complete 3′-UTR of the gene. The DNA sequence of the gene is shown in FIG. 2. The gene sequences determined in preliminary sequencing first showed some gaps in intron 1 and a gap in exon 5 (SEQ ID NOs. 33, 10-15, 34 and 35) which were closed when the complete sequence of the fascin gene (SEQ ID NO. 72) was determined. The last five base pairs of the 3′-UTR of the cDNA sequence were not present in the genomic sequence. The exon/intron boundaries are consistent with the GT/AG rule except for a variation of the 5′-inton boundary of intron 4 (GC instead of GT).

### EXAMPLE 2

### Functional Analysis of the 5′-Flanking Region

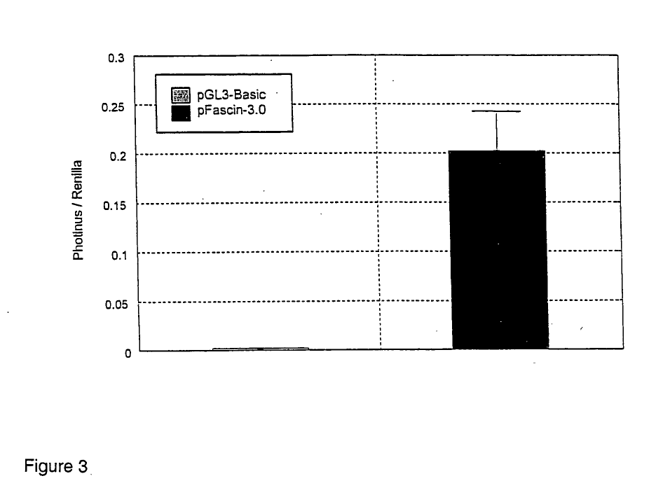

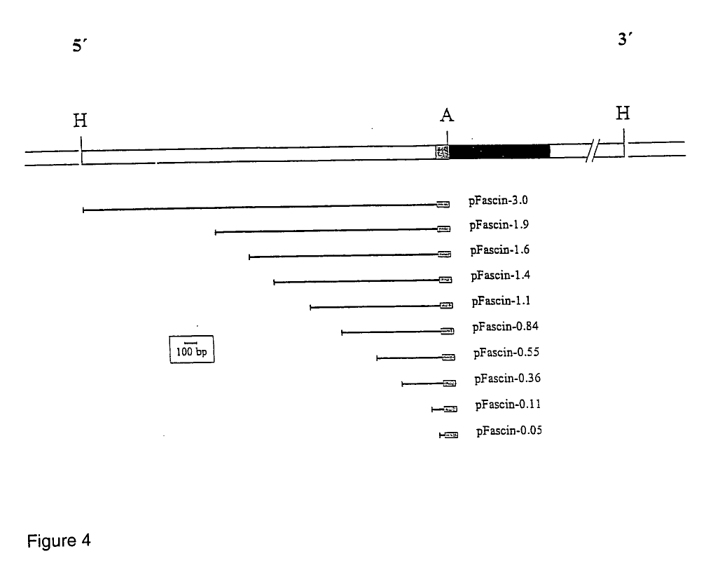

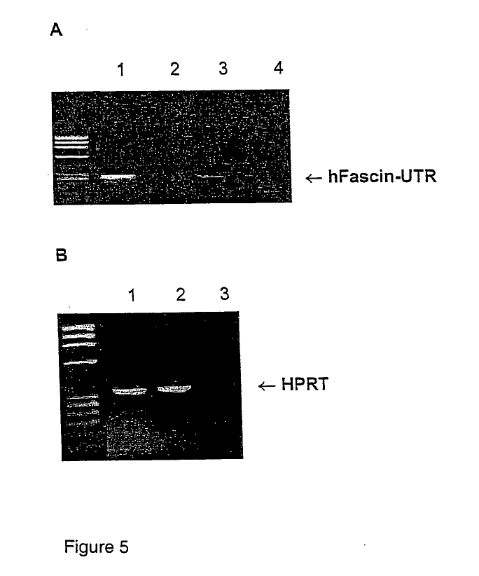

As is typical for a eukaryotic gene promoter, a consensus TATA box motif (TATAAAA) was identified near the transcriptional start. To assess the promoter activity of the 5′-flanking region of the human fascin gene, a promoter-reporter construct (pFascin-3.0, positions 1 to 3069 in SEQ ID NO. 72 and SEQ ID NO. 1, respectively) was constructed, which contained the total length of the isolated 5′-flanking region, and downstream thereby, the first 102 bp of the 5′-non-translated sequence of the fascin gene (position 1 of the published human cDNA clone defined as the transcription start site). Mature dendritic cells which express fascin abundantly, were transiently transfected by biolistic gene transfer of this construct. While the negative control (pGL3-Basic) showed only background luciferase activity, transfection with pFascin-3.0 resulted in a strong reporter expression which was 101-fold increased compared to pGL3-Basic (FIG. 3). To identify cis-acting elements located in the promoter, 5′-deletion clones of pFascin-3.0 were generated (FIG. 4, position 1136 to 3069, 1451 to 3069, 1621 to 3069, 1830 to 3069, 2127 to 3069, 2410 to 3069, 2700 to 3069, 2859 to 3069 and 2915 to 3069 in SEQ ID NO. 72 and SEQ ID NOs. 2 to 8, 21, 22, respectively) and used for transient transfection. The human monocytic cell line THP-1 was used as a negative control, since RT-PCR showed no endogenous fascin expression (FIG. 5).

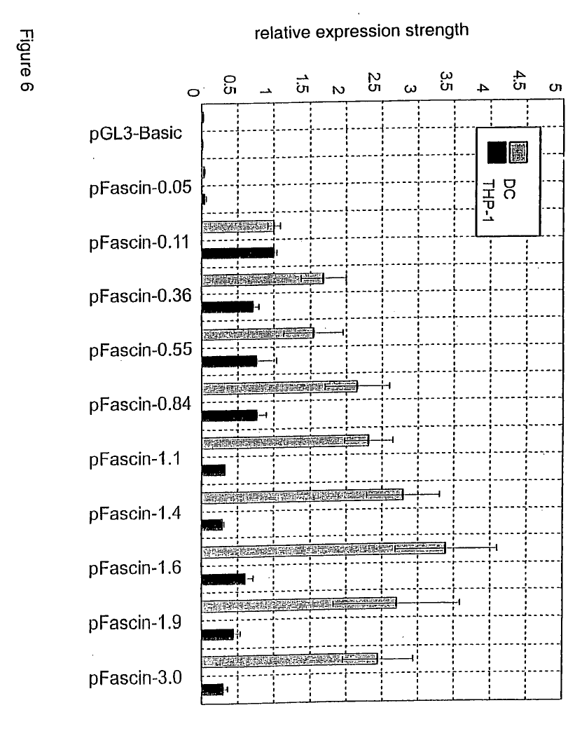

A 5′-deletion construct containing only 53 base pairs of the promoter, including the consensus TATA box (pFascin-0.05, position 2915 to 3069 in SEQ ID NO.72 or SEQ ID NO.22) is not sufficient to bring about luciferase expression (FIG. 6). The first fragment showing a basal promoter activity is a 211 bp fragment containing 109 bp of the promoter (pFascin-0.11, position 2859 to 3069 in SEQ ID NO. 72 and SEQ ID NO. 21, respectively). Transfection with promoter constructs extending to the more distal part of the 5′-flanking sequence resulted in a further stepwise increase of luciferase expression. The highest reporter expression was detected for pFascin-1.6 (position 1451 to 3069 in SEQ ID NO. 72 and SEQ ID NO.3, respectively) which showed a 3.4-fold activity compared with the minimal promoter (pFascin-0.11).

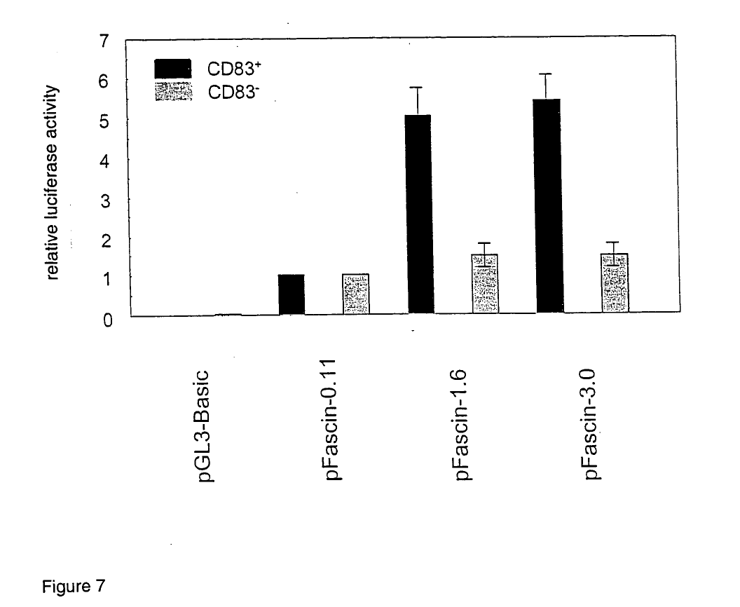

Terminally matured dendritic cells are distinguished from immature dendritic cells by surface expression of marker CD83. The cells of a DC culture were divided into a CD83-positive and a CD83-negative fraction. While in the CD83-positive DC fraction, reporter gene expression for the longer promoter constructs pFascin-1.6 and pFascin-3.0 reached the five-fold value of the basal promoter pFascin-0.11, the expression level in the CD83-negative cell fraction by the use of pFascin-1.6 and pFascin-3.0 was not increased beyond basal expression (FIG. 7). This finding documents that the fascin promoter is predominantly active in terminally matured, CD83-positive dendritic cells. This is particularly advantageous for clinical use.

Surprisingly, the human fascin gene promoter also exhibits a basic activity in THP-1. Therefore, at least one unspecific activating transcriptional element is located in close vicinity 5′ to the TATA-box. However, in contrast to the dendritic cells, the promoter activity in the THP-1 cells decreases as the length of the transfected constructs increases. In the construct with the full promoter length, reporter gene expression is reduced to one-third of the activity detected for construct pFascin-0.11. In summary, within the tested 5′-flanking region of the human fascin gene, DC-specific transcription regulating elements cooperate to enhance expression of the human fascin gene. Additionally, the fascin promoter contains elements which specifically repress transcription in the fascin-negative cell lines, such as THP-1.

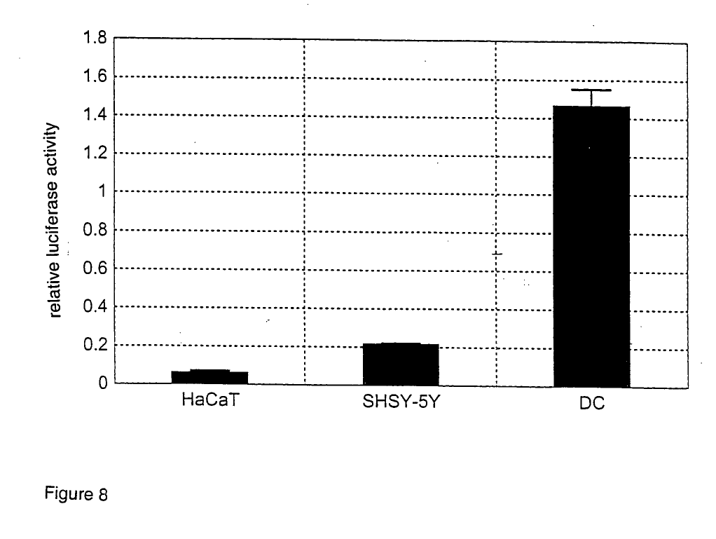

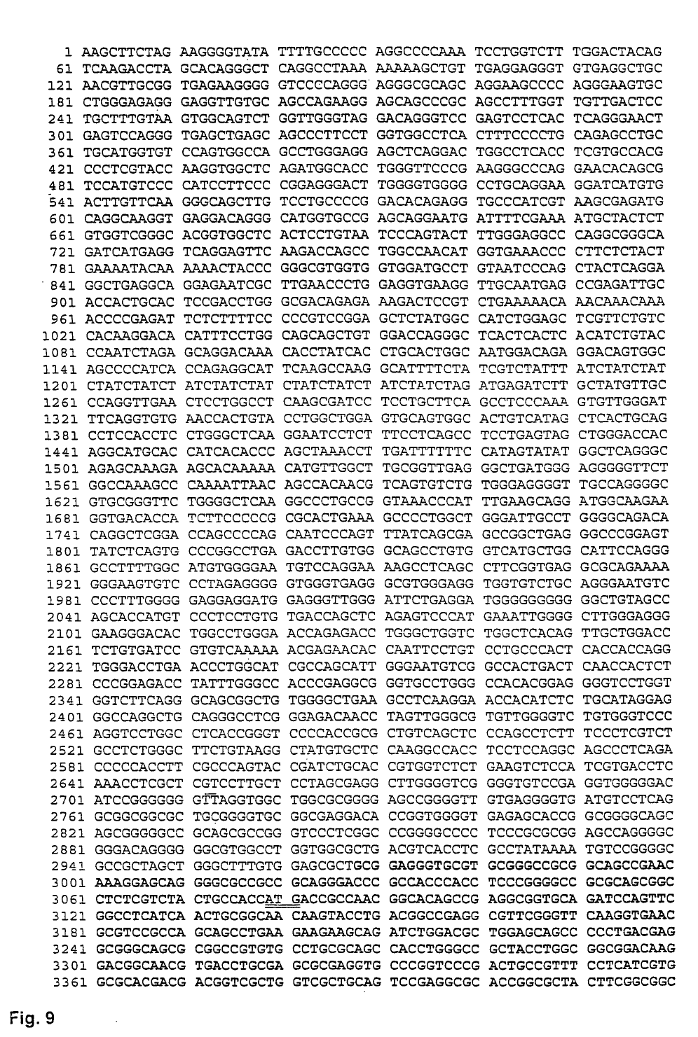

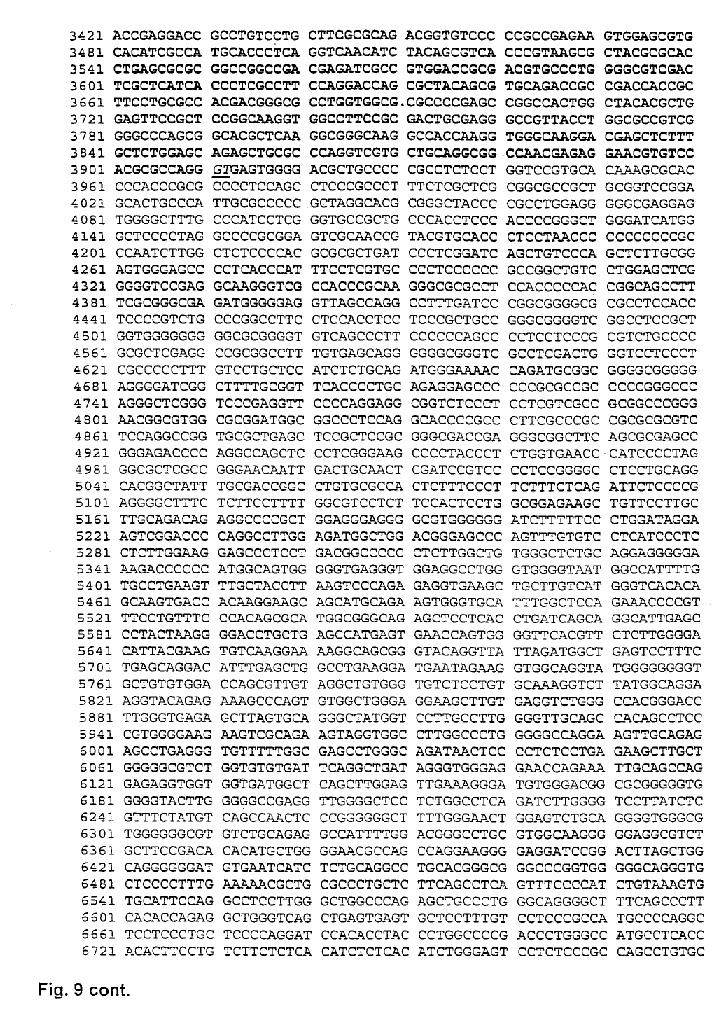

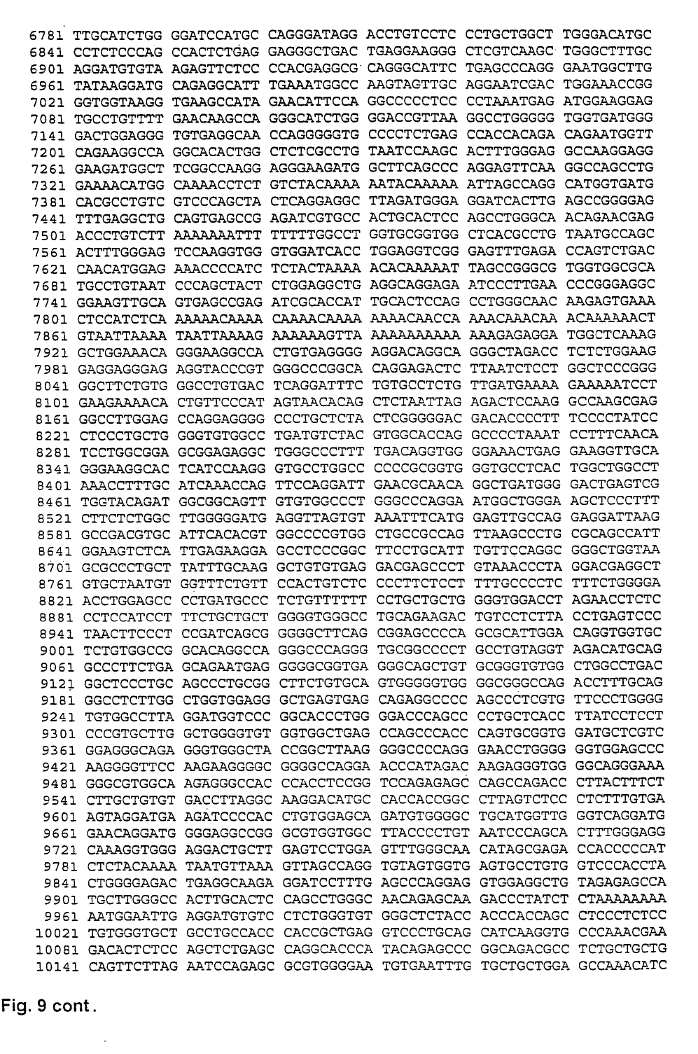

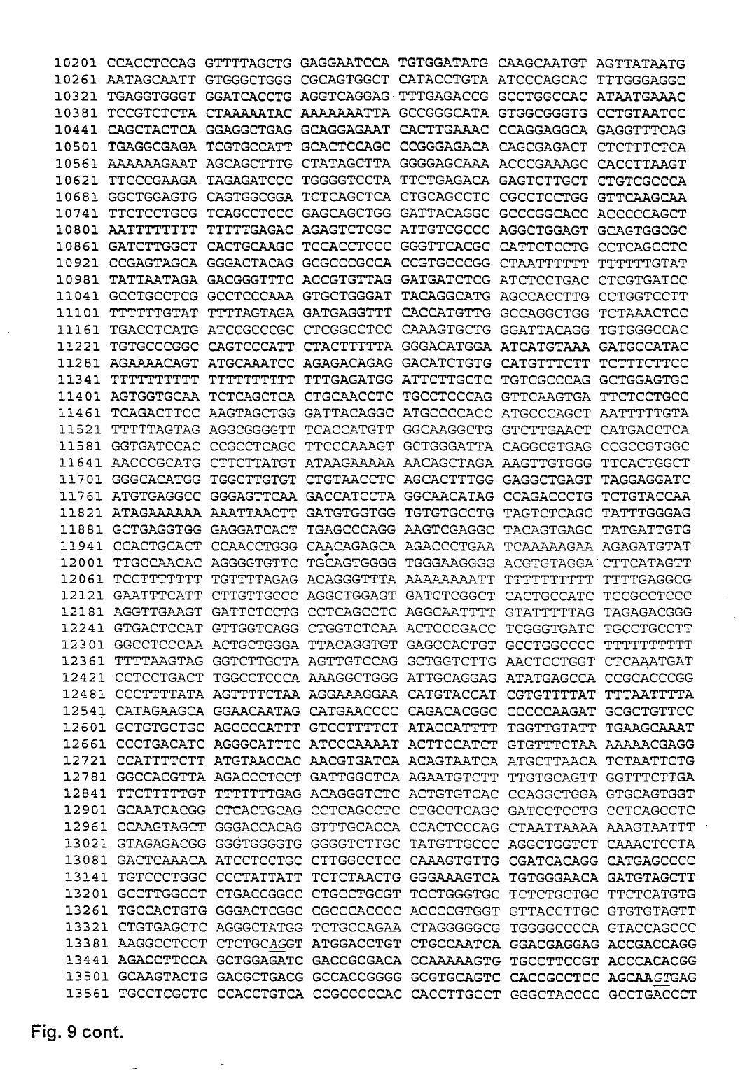

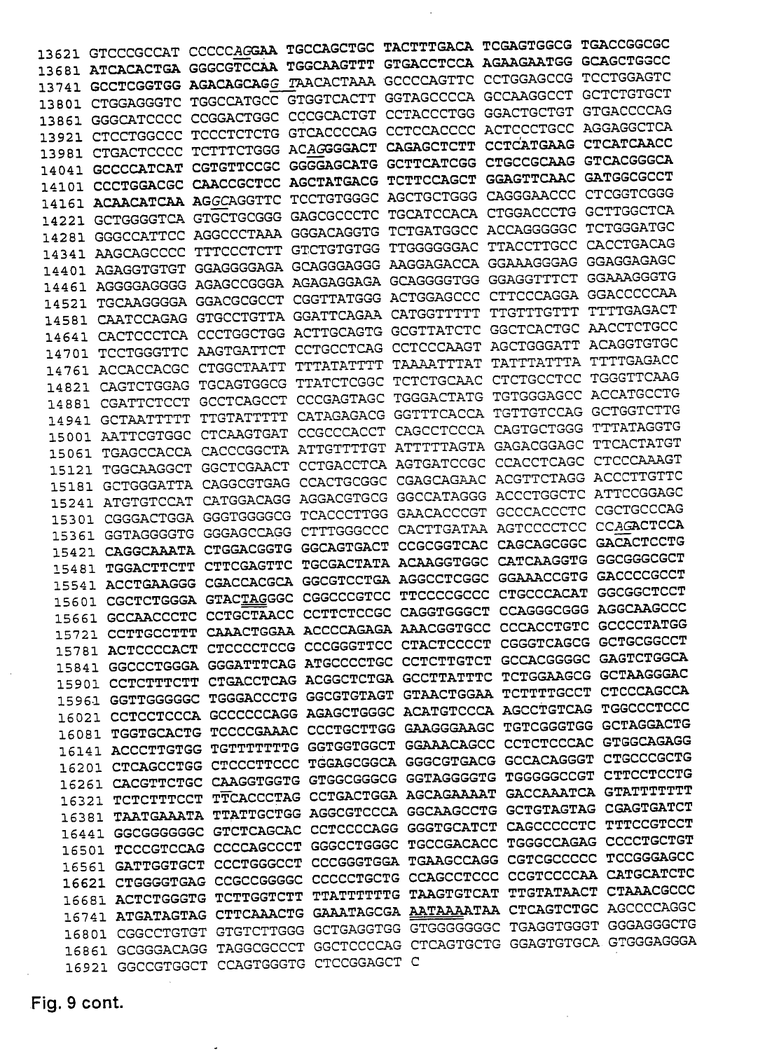

When comparing between different cell-types fascin promoter strength—relative to a positive control which independently from the cell type mediates strong reporter gene expression—the DC specificity of the fascin promoter was confirmed. In the fascin-negative keratinocyte line HaCaT, fascin promoter-dependent reporter activity amounting to less than 10 percent of the positive control only achieved a basal level (FIG. 8). Even in the fascin-positive neuroblastoma line SHSY-5Y, reporter gene expression driven by the fascin promoter only achieved one fifth of the positive control. Compared thereto, the fascin promoter in the strong fascin-positive dendritic cells showed about one and a half-fold strength of the positive control.

1. A regulatory sequence selected from the group consisting of
(a) regulatory sequences comprising the nucleotide sequence indicated under SEQ ID NO. 72 from position 1 to 3069 or under SEQ ID NO. 1;
(b) regulatory sequences comprising the nucleotide sequence contained in the insertion of clone DSM13274 and obtainable by amplification using two oligonucleotides having the sequences indicated under SEQ ID NOs.36 and 37;
(c) regulatory sequences comprising a nucleotide sequence of SEQ ID NO.72 from position 1136 to 3069, 1451 to 3069, 1621 to 3069, 1830 to 3069, 2127 to 3069, 2410 to 3069, or 2700 to 3069 or selected from the group consisting of: SEQ ID NOs. 2 to 8;
(d) regulatory sequences comprising a nucleotide sequence which is contained in the insertion of clone DSM13274 and is obtainable by amplification using a pair of oligonucleotides, the sequences of the oligonucleotides being indicated under the SEQ ID numbers, selected from the group of pairs consisting of 38 and 37; 39 and 37; 40 and 37; 41 and 37; 42 and 37; 43 and 37; and 44 and 37;
(e) regulatory sequences comprising at least a functional part of a sequence indicated in (a) to (d) and causing dendritic cell-specific expression; and
(f) regulatory sequences comprising a nucleotide sequence which hybridizes with a regulatory sequence indicated in (a) to (e), and causing dendritic cell-specific expression.

2. The regulatory sequence according to claim 1 which is combined with at least one of the nucleotide sequences,
a) selected from the group consisting of: the segments of SEQ ID NO. 72 from position 3911 to 13398, 13556 to 13637, 13760 to 14004, 14173 to 15414 and 16791 to 16951 and SEQ ID NOs. 9 to 20, or parts thereof, or
b) contained in the insertion of clone DSM13274 and obtainable by amplification using a pair of oligonucleotides, the sequences of the oligonucleotides being indicated under the SEQ ID numbers, selected from the group of pairs consisting of 45 and 46; 47 and 48, 49 and 50; 51 and 52; 53 and 54; 55 and 56; 57 and 58; 59 and 60; 61 and 62; 63 and 64; 65 and 66; 67 and 68; and 45 and 60.

3. A regulatory sequence according to claim 1 or 2, of human origin.

4. A recombinant nucleic acid molecule containing the regulatory sequence according to any one of claims 1 to 3.

5. A vector containing the regulatory sequence according to any one of claims 1 to 3 or the recombinant nucleic acid molecule according to claim 4.

6. The recombinant nucleic acid molecule according to claim 4 or the vector according to claim 5, which additionally contains a nucleotide sequence to be expressed, wherein expression of the nucleotide sequence is controlled by the regulatory sequence.

7. The recombinant nucleic acid molecule or the vector according to claim 6, wherein the nucleotide sequence encodes an antigen.

8. The recombinant nucleic acid molecule or the vector according to claim 7, wherein the antigen is tumor or pathogen-specific or participates in the formation of Creutzfeldt-Jakob plaques or Alzheimer plaques.

9. The recombinant nucleic acid molecule or the vector according to claim 7, wherein the antigen is an autoantigen or a transplantation antigen.

10. The recombinant nucleic acid molecule or the vector according to claim 7, wherein the antigen is an allergen.

11. The recombinant nucleic acid molecule or the vector according to claim 6, wherein the nucleotide sequence encodes a protein which regulates an immune response.

12. The recombinant nucleic acid molecule or the vector according to claim 11, wherein the protein is a cytokine or a co-stimulatory molecule.

13. The recombinant nucleic acid molecule or the vector according to claim 11, wherein the regulation is inhibition.

14. The recombinant nucleic acid molecule or the vector according to claim 11, 12 or 13, wherein the protein is IL-10 or TGF-β.

15. The recombinant nucleic acid molecule or the vector according to claim 11 or 12, wherein the regulation is an increase of the immune response.

16. The recombinant nucleic acid molecule or the vector according to claim 11, 12 or 15, wherein the protein is IL-2, IL-4, IL-12, IL-15, IL-18, IFN-gamma, IFN-alpha, DC-CK1, MDC or GM-CSF.

17. The recombinant nucleic acid molecule or the vector according to claim 11, 12 or 15, wherein the protein is a member of the B7 family, ICOS ligand or CD40.

18. The recombinant nucleic acid molecule or the vector according to claim 6, wherein the nucleotide sequence encodes an apoptosis-inducing molecule.

19. The recombinant nucleic acid molecule or the vector according to claim 18, wherein the apoptosis-inducing molecule belongs to the TNF superfamily.

20. The recombinant nucleic acid molecule or the vector according to claim 6, wherein the nucleotide sequence is an antisense sequence or expresses a ribozyme.

21. The recombinant nucleic acid molecule or the vector according to claim 20, wherein the antisense sequence or the ribozyme is specific for an mRNA encoding a cytokine or a co-stimulatory molecule.

22. The recombinant nucleic acid molecule or the vector according to claim 6, wherein the nucleotide sequence encodes a transcription factor.

23. The recombinant nucleic acid molecule or the vector according to claim 6 which additionally contains a second nucleotide sequence to be expressed, wherein one nucleotide sequence encodes an antigen and the second nucleotide sequence encodes a protein which regulates an immune response.

24. The vector according to any one of claims 5 to 23, which is a virus.

25. The vector according to any one of claims 6 to 24, which is suitable for gene therapy or DNA vaccination.

26. The recombinant nucleic acid molecule or the vector according to claim 6, wherein the nucleotide sequence is a reporter gene.

27. A method for preparing genetically modified host cells, characterized in that the host cells are transfected with a vector according to any one of claims 5 to 26 and the transfected host cells are cultured in a culture medium.

28. A host cell which is genetically modified with the regulatory sequence according to any one of claims 1 to 3, the recombinant nucleic acid molecule according to any one of claims 4, 6 to 23 and 26 or the vector according to any one of claims 5 to 26, or obtainable by the method according to claim 27.

29. The host cell according to claim 28, which is a dendritic cell.

30. The host cell according to claim 28 or 29, of human origin.

31. A nucleotide sequence comprising a fragment having a length of at least 15 nucleotides which specifically hybridizes with a strand of a regulatory sequence according to any one of claims 1 to 3 under stringent conditions.

32. A method for the antigen-specific stimulation of T cells in vitro, comprising the steps of:
(a) transfecting dendritic cells with a vector according to any one of claims 7 to 10, alone or in combination with a vector according to any one of claims 11, 12, 15, 16, 17, 20 and 22, or with a vector according to claim 23;
(b) co-culturing the transfected dendritic cells obtained in step (a) with T-cells; and
(c) detecting the activation of the T cells of step (b).

33. A method for preparing a pharmaceutical composition, which comprises steps (a) to (c) according to claim 32 and additionally the step of
(d) formulating a pharmaceutical composition by mixing the stimulated T-cells obtained in step (c) with a pharmaceutically acceptable carrier.

34. A method for the in vitro preparation of T cell-stimulating dendritic cells, comprising the steps of
(a) transfecting dendritic cells with a vector according to any one of claims 7 to 10, alone or in combination with a vector according to any one of claims 11 or 22, or with a vector according to claim 23; and
(b) culturing the transfected dendritic cells in a suitable medium and/or detecting the T-cell stimulating activity.

35. A method for preparing a pharmaceutical composition, which comprises steps (a) and
(b) according to claim 34 and additionally the step of
(c) formulating a pharmaceutical composition by mixing the T cell-stimulating dendritic cells obtained in step (b) with a pharmaceutically acceptable carrier.

36. The method according to any one of claims 32 to 35, wherein the dendritic cells are of human origin.

37. A pharmaceutical composition comprising the recombinant nucleic acid molecule according to any one of claims 6 to 23, the vector according to any one of claims 6 to 25, the host cell according to any one of claims 28 to 30, antigen-specifically stimulated T-cells obtainable by the method according to claim 32, or T cell-stimulating dendritic cells obtainable by the method according to claim 34, and optionally a pharmaceutically acceptable carrier.

38. The pharmaceutical composition according to claim 37, which is a vaccine.

39. Use of the recombinant nucleic acid molecule according to any one of claims 6 to 8 and 10, the vector according to any one of claims 6 to 8, 10, 24 and 25, alone or in combination with a recombinant nucleic acid molecule or vector according to any one of claims 11, 12, 14 to 17 and 22, the host cell according to any one of claims 28 to 30, the recombinant nucleic acid molecule or vector according to claim 23, antigen-specially stimulated T-cells obtainable by the method according to claim 32, or T cell-stimulating dendritic cells obtainable by the method according to claim 34, for preparing a pharmaceutical composition for the vaccination against viruses, bacteria, fungi, parasites, tumors, allergens, Creutzfeldt-Jakob plaques or Alzheimer plaques or for the gene therapeutic treatment of tumors or viral, bacterial or parasitic infections or allergies.

40. Use of the recombinant nucleic acid molecule according to any one of claims 6, 7, 9 and 10 or the vector according to any one of claims 6, 7, 9, 10, 24 and 25, alone or in combination with a recombinant nucleic acid molecule or a vector according to any one of claims 11, 13, 14, 18 to 22, of the nucleic acid molecule or the vector according to claim 23, or of the host cell according to any one of claims 28 to 30, for preparing a pharmaceutical composition for treating autoimmune diseases, graft rejection or allergies.

41. Use of the recombinant nucleic acid molecule according to claim 18 or 19 or the vector according to any one of claims 18, 19, 24 and 25 for preparing a pharmaceutical composition for preventing graft rejection and autoimmune reactions.

42. Use of the regulatory sequence according to any one of claims 1 to 3, the recombinant nucleic acid molecule according to any one of claims 4, 6 to 23 or the vector according to any one of claims 5 to 25 for specifically expressing antigens or immunoregulatory proteins in dendritic cells.

43. Use of the regulatory sequence according to any one of claims 1 to 3, the recombinant nucleic acid molecule according to claim 4, 6 or 26 or the vector according to claim 5, 6 or 26 for identifying and isolating cis-elements from the regulatory sequence which mediate expression specific to dendritic cells.

44. Use of the regulatory sequence according to any one of claims 1 to 3, the recombinant nucleic acid molecule according to claim 4, 6 or 26 or the vector according to claim 5, 6 or 26 for determining the degree of maturation of dendritic cells.

45. Use of the regulatory sequence according to any one of claims 1 to 3, the recombinant nucleic acid molecule according to claim 4, 6 or 26 or the vector according to claim 5, 6 or 26 for identifying and isolating factors which mediate expression specific to dentritic cells.

46. Use of the regulatory sequence according to claim 1 or parts thereof for blocking transcription factors by providing transcription factor binding sites in dendritic cells.
